(19) United States
(12) Patent Application Publication
(10) Pub. No.: US 2020/0395123 A1
(43) Pub. Date: Dec. 17, 2020

(54) SYSTEMS AND METHODS FOR PREDICTING LIKELIHOOD OF MALIGNANCY IN A TARGET TISSUE

(71) Applicant: International Business Machines Corporation, Armonk, NY (US)

(72) Inventors: AYELET AKSELROD-BALLIN, TEL-AVIV (IL); MICHAL CHOREV, TEL-AVIV (IL); ALON HAZAN, TORONTO (CA); ROIE MELAMED, HAIFA (IL); YOEL SHOSHAN, HAIFA (IL); ADAM SPIRO, TEL-AVIV (IL)

(21) Appl. No.: 16/442,510

(22) Filed: Jun. 16, 2019

(51) Int. Cl. G16H 50/20; G16H 50/50; G16H 30/40

(52) U.S. Cl. CPC G16H 50/20; G16H 30/40; G16H 50/50

(57) ABSTRACT

There is provided, a method of selecting patients for treatment, comprising: feeding anatomical image(s) of a patient depicting a target tissue, and non-imaging clinical parameters of the patient into neural network component(s) of a model, outputting by the neural network component(s), an intermediate vector storing a plurality of embedding values computed for the anatomical image(s), a plurality of values outputted by a dense layer of the neural network component(s) in response to an input of at least some of the non-imaging clinical parameters, and an intermediate value indicative of likelihood of malignancy for the target tissue, feeding into a classifier component of the model, a feature vector created from the intermediate vector and the plurality of non-imaging clinical parameters, and selecting patients for treatment according to an indication of likelihood of malignancy in the target tissue outputted by the model.

### BACKGROUND

The present invention, in some embodiments thereof, relates to treatment of a patient based on likelihood of malignancy and, more specifically, but not exclusively, to systems and methods for computing likelihood of malignancy using a model.

Cancer is a leading cause of death. Traditionally, patients are screened for cancer using anatomical imaging (e.g., mammography, CT scan, x-ray). The radiologist manually evaluates the images to determine likelihood of cancer. A biopsy of the suspected tissue (e.g., lesion) may be performed to confirm the presence of cancer, or exclude the cancer diagnosis. Due to the heavy burden on radiologists, and unnecessarily performed biopsies (i.e., where cancer is not present), automated processes are being developed to aid in diagnosis of cancer. However, the accuracy of such automated processes are still not good enough to enable relying on their results in clinical practice.

### SUMMARY

According to a first aspect, a method of selecting patients for treatment, comprises: feeding at least one anatomical image of a patient depicting a target tissue, and a plurality of non-imaging clinical parameters of the patient into at least one neural network component of a model, outputting by the at least one neural network component, an intermediate vector storing a plurality of embedding values computed for the at least one anatomical image, a plurality of values outputted by a dense layer of the at least one neural network component in response to an input of at least some of the non-imaging clinical parameters, and an intermediate value indicative of likelihood of malignancy for the target tissue, feeding into a classifier component of the model, a feature vector created from the intermediate vector and the plurality of non-imaging clinical parameters, and selecting patients for treatment according to an indication of likelihood of malignancy in the target tissue outputted by the model.

According to a second aspect, a method of training a model used for selecting patients for treatment, comprises: training at least one neural network component of the model for outputting an intermediate value indicative of likelihood of malignancy for a target tissue of a target patient in response to an input of at least one anatomical image depicting a target tissue of the target patient, and at least some non-imaging clinical parameters of the target patient, according to a training dataset storing, for each of a plurality of sample patients, a ground truth indication of malignancy, at least one anatomical image depicting the target tissue, and value for the plurality of non-imaging clinical parameters, creating an intermediate training dataset storing a respective feature vector for each of the plurality of sample patients, wherein each feature vector is created from a respective intermediate vector and the plurality of non-imaging clinical parameters for the respective sample individual, wherein each respective intermediate vector stores a plurality of embedding values computed for the at least one anatomical image of the respective sample individual and a plurality of values, outputted by a dense layer of the trained at least one neural network component in response to an input of at least some of the non-imaging clinical parameters of the respective sample individual, and an intermediate value indicative of likelihood of malignancy for the target tissue of the sample individual, training a classifier component of the model according to feature vectors stored in the intermediate training dataset and corresponding ground truth indications of malignancy, and providing the model including the trained at least one neural network component and the trained classifier component for selecting patients for treatment according to a computed indication of likelihood of malignancy in a target tissue of a target patient outputted by the model.

According to a third aspect, a system for selecting patients for treatment, comprises: at least one hardware processor executing a code for: feeding at least one anatomical image of a patient depicting a target tissue, and a plurality of non-imaging clinical parameters of the patient into at least one neural network component of a model, outputting by the at least one neural network component, an intermediate vector storing a plurality of embedding values computed for the at least one anatomical image, a plurality of values outputted by a dense layer of the at least one neural network component in response to an input of at least some of the non-imaging clinical parameters, and an intermediate value indicative of likelihood of malignancy for the target tissue, feeding into a classifier component of the model, a feature vector created from the intermediate vector and the plurality of non-imaging clinical parameters, and selecting patients for treatment according to an indication of likelihood of malignancy in the target tissue outputted by the model.

In a further implementation of the first, second, and third aspects, treatment comprises a biopsy of the target tissue.

In a further implementation of the first, second, and third aspects, selecting comprises tagging a record of the patient with an indication of recommendation for treatment, for patients having a value indicative of likelihood of malignancy above a threshold.

In a further implementation of the first, second, and third aspects, the at least one neural network comprises a first and second subset, wherein the at least one anatomical image and the plurality of non-imaging clinical parameters are fed into the first subset and the at least one anatomical image is fed into the second subset, wherein the intermediate vector stores outputs of the first and second subsets, wherein the first subset outputs the plurality of embedding values, the plurality of values outputted by the dense layer, and the intermediate value, and the second subset outputs a second plurality of embedding values computed for the at least one anatomical image and a second intermediate value indicative of likelihood of malignancy for the target tissue.

In a further implementation of the first, second, and third aspects, the at least one anatomical image comprises a plurality of anatomical images, wherein each one of the plurality of anatomical images is fed into the at least one neural network component, wherein outputs of the at least one neural network for the plurality of anatomical images are aggregated to compute the intermediate vector.

In a further implementation of the first, second, and third aspects, each of the plurality of anatomical images depicts a respective unique image planes of the target tissue.

In a further implementation of the first, second, and third aspects, the at least one anatomical image comprises a plurality of anatomical images each fed into the at least one neural network component, the feature vector further includes a plurality of metadata values computed for pairs of the plurality of anatomical images according to relationships between respective likelihood of malignancy computed by the at least one neural network component.

In a further implementation of the first, second, and third aspects, the relationships are computed for a first and second image of each pair of the plurality of anatomical images, selected from the group consisting of: likelihood of malignancy for the first image divided by a sum of likelihood of malignancy for the first image and likelihood of malignancy for the second image, absolute value of a difference between likelihood of malignancy for the first image and likelihood of malignancy for the second image, and maximum of the likelihood of malignancy for the first image and likelihood of malignancy for the second image.

In a further implementation of the first, second, and third aspects, the intermediate vector is computed as an output of a last fully connected layer that receives a concatenation of an output of a sub-component of the at least one neural network that is fed the at least one anatomical image, and an output of the dense layer of the at least one neural network that is fed at least some of the non-imaging clinical parameters.

In a further implementation of the first, second, and third aspects, the intermediate value is outputted by an output layer of the at least one neural network that is fed the at least one anatomical image and the plurality of non-imaging clinical parameters.

In a further implementation of the first, second, and third aspects, the at least some of the non-imaging clinical parameters are selected according to a training dataset of a plurality of sample patients, according to a statistical correlation between each non-imaging clinical parameter and a ground truth indicative of a diagnosis of malignancy.

In a further implementation of the first, second, and third aspects, the at least one neural network comprises an ensemble of a plurality of neural networks each differing by at least one neural network parameter, wherein the at least one anatomical image comprises a plurality of anatomical images each fed into each neural network of the ensemble, wherein the intermediate vector is computed as an aggregation of the plurality of embedding values computed by the ensemble and an aggregation of a plurality of values outputted by each respective dense layers of the ensemble.

In a further implementation of the first, second, and third aspects, the non-imaging clinical parameters exclude an external manual and/or automatic analysis of the at least one anatomical image.

In a further implementation of the first, second, and third aspects, the indication of likelihood of malignancy in the target tissue comprises indication of likelihood of malignancy in breast tissue.

In a further implementation of the first, second, and third aspects, the non-imaging clinical parameters are selected from the group consisting of: demographics, age, last body mass index, maximum body mass index, last body mass index class, maximum body mass index class, gynecological history, age at first menstruation, age at last menstruation, indication of postmenopausal, number of menstruation years, pregnancies count, past pregnancies, indication of is breastfeeding, number of children breastfed, indication of current use of hormone replacement therapy, cancer history, family breast cancer first degree, family breast or ovarian cancer, number of relatives with breast or ovarian cancer, minimum age in family for cancer, any personal cancer history, symptoms, lump complaint by woman, bilateral lump complaint by woman, lump complaint by woman in the past, pain complaint by woman, bilateral pain complaint by woman, breast radiology history, past number of breast imaging encounters, past breast density, past final BIRADS assessment DM left, past final BIRADS assessment DM right, past final BIRADS assessment US left, past final BIRADS assessment US right.

In a further implementation of the first, second, and third aspects, further comprising defining the at least some of the non-imaging clinical parameters based on the training dataset by computing a statistical correlation between each non-imaging clinical parameter and ground truth indication of malignancy, and selecting the at least some of the non-imaging clinical parameters according to a requirement of the statistical correlation.

In a further implementation of the third aspect, the system further comprises a code for: training the at least one neural network component of the model for outputting the intermediate value indicative of likelihood of malignancy for the target tissue of the target patient, according to a training dataset storing, for each of a plurality of sample patients, a ground truth indication of malignancy, at least one anatomical image depicting the target tissue, and value for the plurality of non-imaging clinical parameters, creating an intermediate training dataset storing a respective feature vector for each of the plurality of sample patients, wherein each feature vector is created from a respective intermediate vector and the plurality of non-imaging clinical parameters for the respective sample individual, wherein each respective intermediate vector stores a plurality of embedding values computed for the at least one anatomical image of the respective sample individual and a plurality of values, outputted by a dense layer of the trained at least one neural network component in response to an input of at least some of the non-imaging clinical parameters of the respective sample individual, and an intermediate value indicative of likelihood of malignancy for the target tissue of the sample individual, and training the classifier component of the model according to feature vectors stored in the intermediate training dataset and corresponding ground truth indications of malignancy, wherein the model includes the trained at least one neural network component and the trained classifier component.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method for computing an indication of likelihood of malignancy in a target tissue of a patient by a trained model based on a combination of images and non-imaging clinical data, in accordance with some embodiments of the present invention;

FIG. 2 is a block diagram of components of a system for computing an indication of likelihood of malignancy in a target tissue of a patient by a trained model and/or for training the model based on a combination of images and non-imaging clinical data, in accordance with some embodiments of the present invention;

FIG. 3 is a flowchart of a method of training the model for computing the indication of likelihood of malignancy in the target tissue of the patient based on a combination of images and non-imaging clinical data, in accordance with some embodiments of the present invention;

FIG. 4 is a schematic depicting an exemplary architecture of a model for outputting an indication of likelihood of malignancy in a target tissue based on a combination of one or more images and non-imaging clinical parameters, in accordance with some embodiments of the present invention;

FIG. 5 is a block diagram of an exemplary architecture of a neural network component of a model for outputting an indication of likelihood of malignancy in a target tissue based on a combination of one or more images and non-imaging clinical parameters, in accordance with some embodiments of the present invention; and

FIGS. 6A-6L are tables presenting results from an evaluation experiment performed by inventions, in accordance with some embodiments of the present invention.

### DETAILED DESCRIPTION

The present invention, in some embodiments thereof, relates to treatment of a patient based on likelihood of malignancy and, more specifically, but not exclusively, to systems and methods for computing likelihood of malignancy using a model.

It is noted that some parts of this patent application are in press, and scheduled to be published on Jun. 16, 2019, under: Akselrod-Balin, A., Chorev M., et al. (in press). Predicting Breast Cancer by Applying Deep Learning to Linked Health Records and Mammography Images. Radiology, the contents of which are also incorporated in their entirety by reference into the specification.

An aspect of some embodiments of the present invention relates to systems, methods, an apparatus, and/or code instructions for a model for outputting an indication of likelihood of malignancy in a target tissue based on a combination of anatomical image(s) and non-imaging clinical data fed into the model. Anatomical image(s) (e.g., CT, MRI, x-ray, mammography, ultrasound, PET) of a patient depicting a target tissue, and non-imaging clinical parameters of the patient, are fed into a neural network component(s) of a model. The neural network component(s) output an intermediate vector, which includes embedding values computed for the anatomical image(s), values outputted by a dense layer of the neural network component in response to an input of at least some of the non-imaging clinical parameters, and an intermediate value indicative of likelihood of malignancy for the target tissue. A feature vector is created from the intermediate vector and the non-imaging clinical parameters. The feature vector is fed into a classifier component of the model. The model outputs an indication of likelihood of malignancy in the target tissue. Patients may be selected for treatment according to the output of the model. For example, the model may output likelihood of breast cancer. Patients having risk values above a threshold may be selected for biopsy.

At least some implementations of the systems, methods, apparatus, and/or code instructions described herein address the medical problem of estimating likelihood of malignancy in a target tissue of a patient, based on non-invasive testing, for example, anatomical images and/or non-imaging clinical data (e.g., obtained from an electronic medical record of the patient). At least some implementations of the systems, methods, apparatus, and/or code instructions described herein improve the medical process of planning treatment of patient based on likelihood of malignancy in a target tissue determined based on non-invasive testing. The likelihood of malignancy is used to determine further treatment of the patient, for example, whether a biopsy of the target tissue is performed or not. For example, when likelihood of malignancy is high, a biopsy may be performed to confirm malignancy. When likelihood of malignancy is low, the biopsy may be delay or not performed. The patient may be monitored to determine whether malignancy is developing or not.

At least some implementations of the systems, methods, apparatus, and/or code instructions described herein improve the technological field of models and/or statistical classifiers and/or neural networks that compute likelihood of malignancy. The technological improvement arises, at least in part, from the described architecture of the model and/or neural network component and/or sub-classifier component, that integrate anatomical imaging data with non-imaging clinical parameters (e.g., obtained from an EMR of the patient) for computing likelihood of malignancy in the target tissue. The integration of anatomical imaging data with non-imaging clinical parameters is achieved, at least in part, by an intermediate vector that is outputted by a neural network component(s) of a model. The intermediate vector stores embedding values computed for the anatomical image(s), values outputted by a dense layer of the neural network component in response to an input of at least some of the non-imaging clinical parameters, and an intermediate value indicative of likelihood of malignancy for the target tissue. A feature vector crated from the intermediate vector the non-imaging clinical parameters is fed into a classifier component of the model.

The model implemented by at least some of the systems, methods, apparatus, and/or code instructions described herein provides an accuracy of detecting malignancy in target tissue that is comparable to radiologists as defined by the American benchmark for screening digital mammography (75% sensitivity and 88%-95% specificity). Details of an experimental and/or computational evaluation conducted by Inventors that the model described herein provides high accuracy of detecting malignancy is described below in the “Examples” section. A high accuracy in computing likelihood of malignancy using non-invasive testing reduces likelihood of cancer misdiagnosis, and/or spares patients from unneeded biopsies and/or other invasive procedures when risk of malignancy computed from the non-invasive testing is low.

At least some of the systems, methods, apparatus, and/or code instructions described herein improve the medical process of determining which patients who underwent breast imaging are to have a biopsy for evaluation of breast cancer. The improvement is at least due to a high accuracy of likelihood of breast cancer based on imaging and non-imaging clinical parameters computed by the model described herein, for example, accuracy comparable to a human radiologist.

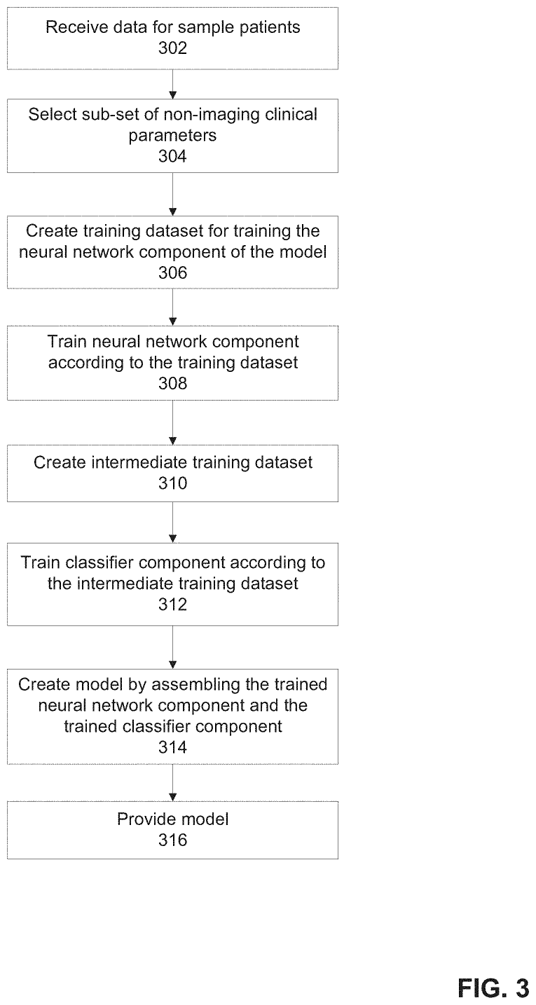

Breast cancer is the second leading cause of cancer-related deaths, and the most commonly diagnosed cancer among women across the world, for example, as described with reference to Society AC. Global Cancer Facts & Figures. 3rd Edition. Atlanta: American Cancer Society; 2015. Digital mammography (DM) is the primary imaging modality for breast cancer screening, both for screening asymptomatic women and in a diagnostic workup setting, for example, as described with reference to Giger M L, Karssemeijer N, Schnabel J A. Breast image analysis for risk assessment, detection, diagnosis, and treatment of cancer. Annual review of biomedical engineering. 2013; 15:327-357, and has been shown to reduce breast cancer mortality, for example, as described with reference to Kopans D B. Beyond randomized controlled trials: Organized mammographic screening substantially reduces breast carcinoma mortality. Cancer. 2002; 94 (2):580-581. doi:10.1002/cncr.10220. In standard current practice, a radiologist evaluates mammograms, and classifies the findings according to the American College of Radiology Breast Imaging-Reporting and Data System (BI-RADS) Lexicon, for example, as described with reference to Sickles E A, D'Orsi CJ, Bassett L W. American College of Radiology Breast Imaging Reporting and Data System Atlas (ACR BI-RADS Atlas). Reston, Va.: American College of Radiology; 2013. An abnormal finding detected in a DM typically requires a diagnostic workup, which may include additional views or other modality. If a lesion is suspicious, further evaluation with a biopsy is recommended. Image analysis is challenging due to the fine-grained differences between lesions and background in the large DM images, the wide range of abnormalities, the non-rigid nature of the breast, and the small number of cancers in a screening population of average risk women, for example, as described with reference to Giger M L, Karssemeijer N, Schnabel J A. Breast image analysis for risk assessment, detection, diagnosis, and treatment of cancer. Annual review of biomedical engineering. 2013; 15:327-357. This leads to substantial intra-observer and interobserver variability, for example, as described with reference to Katalinic A, Bartel C, Raspe H, Schreer I. Beyond mammography screening: quality assurance in breast cancer diagnosis (The QuaMaDi Project). British journal of cancer. 2007; 96 (1):157. The average performance measures for radiologist's screening are reported at 86.9% sensitivity and 88.9% specificity, for example, as described with reference to Lehman C D, Arao R F, Sprague B L, et al. National performance benchmarks for modern screening digital mammography: update from the Breast Cancer Surveillance Consortium. Radiology. 2016; 283 (1):49-58. The abnormal interpretation rate is 11.6%, and the cancer detection rate is 0.51% with a false negative (FN) rate of 0.08%.

Breast cancer risk prediction models based on clinical features assist physicians in estimating the probability of an individual or a population to develop breast cancer within certain timeframes. In a systematic survey of risk prediction models, Meads C, Ahmed I, Riley R D. A systematic review of breast cancer incidence risk prediction models with meta-analysis of their performance. Breast cancer research and treatment. 2012; 132 (2):365-377. doi:10.1007/s10549-011-1818-2 reported a limited performance when applied to general populations (AUC of up to 0.67 [0.65-0.68]), with improved abilities on high-risk populations (AUC of 0.76 [0.70-0.82]).

Recently, machine learning (ML) and its sub-discipline deep learning (DL) have obtained remarkable results in the healthcare domain, for example, as described with reference to Gulshan V, Peng L, Coram M, et al. Development and validation of a deep learning algorithm for detection of diabetic retinopathy in retinal fundus photographs. Jama. 2016; 316 (22):2402-2410, Esteva A, Kuprel B, Novoa R A, et al. Dermatologist-level classification of skin cancer with deep neural networks. Nature. 2017; 542 (7639):115, Bejnordi B E, Veta M, Van Diest P J, et al. Diagnostic assessment of deep learning algorithms for detection of lymph node metastases in women with breast cancer. Jama. 2017; 318 (22):2199-2210, Rajpurkar P, Irvin J, Zhu K, et al. CheXNet: Radiologist-Level Pneumonia Detection on Chest X-Rays with Deep Learning. arXiv:171105225 [cs, stat]. November 2017. http://arxiv(dot)org/abs/1711.05225. Accessed Sep. 10, 2018, Litjens G, Kooi T, Bejnordi B E, et al. A survey on deep learning in medical image analysis. Medical Image Analysis. 2017;42:60-88. doi:10.1016/j.media.2017.07.005, Lehman C D, Yala A, Schuster T, et al. Mammographic Breast Density Assessment Using Deep Learning: Clinical Implementation. Radiology. 2018; 290 (1):52-58. doi:10.1148/radiol.2018180694. Computational models based on deep neural network (DNN) are increasingly used to analyze healthcare data. Still, the efficacy of traditional computer-aided detection (CAD) systems is a topic of controversy, for example, as described with reference to Gilbert F J, Astley S M, Gillan M G, et al. Single reading with computer-aided detection for screening mammography. New England Journal of Medicine. 2008; 359 (16):1675-1684, van Ginneken B, Schaefer-Prokop C M, Prokop M. Computer-aided diagnosis: how to move from the laboratory to the clinic. Radiology. 2011; 261 (3):719-732, Bahl M. Detecting Breast Cancers with Mammography: Will AI Succeed Where Traditional CAD Failed? Radiology. 2018; 290 (2):315-316. doi:10.1148/radiol.2018182404. However, as described herein, the architecture of the model described herein computes likelihood of malignancy based on imaging and non-imaging clinical data with accuracy comparable to a human radiologist, and therefore applicable for clinical practice.

As described in the “Examples” section below, Inventors performed an experiment for evaluation of the ability of the model described herein to compute likelihood of breast cancer. A summary of the experiment is now provided. Full details are discussed below in the “Examples” section.

In the experimental evaluation, the model described herein was able to correctly identify cancer in 34 of the 71 (48%) women where initial radiologist diagnosis was negative, but cancer was detected within a year, which provides additional evidence that the model described herein improves accuracy over manual radiology reading alone. Moreover, clinical risk prediction was improved over Gail's (p<0.004)—Gail M H, Brinton L A, Byar D P, et al. Projecting Individualized Probabilities of Developing Breast Cancer for White Females Who Are Being Examined Annually. JNCI Journal of the National Cancer Institute. 1989; 81 (24):1879-1886. doi:10.1093/jnci/81.24.1879, by adding clinical features associated with breast cancer to those commonly used in risk estimation. Those features included readily available white blood cells, endocrinological, and immune system characteristics.

Materials and Methods: In the retrospective study, a dataset of 52,936 images was collected from 13,234 women who underwent at least one mammogram between 2013 and 2017 and had health records for at least one year prior to the mammogram. An implementation of the model described herein was trained on 9,611 women for two key breast cancer prediction tasks: prediction of biopsy malignancy and differentiation of normal from abnormal screening examinations. Association of features with outcomes was estimated using t-test and Fisher exact test. Model comparisons were performed using 95% confidence interval or using DeLong test.

Results: The implementation of the model described herein was validated on 1,055 women and tested on 2,548, with mean age of 55±10. The implementation of the model described herein successfully identified 34 out of 71 false negative patients in the test set based on radiologist reports (48%, 95% confidence interval (CI) ±0.002). For the malignancy prediction task, the implementation of the model described herein obtained area under the receiver operating characteristic curve (AUC) of 0.91 (95% CI, ±0.002). The performance of the implementation of the model described herein was comparable to radiologists' performance as reported in the American national benchmark for screening digital mammography (75% sensitivity and 88%-95% specificity). When trained on clinical data alone, the implementation of the model described herein performed better than Gail's (p<0.004).

As discussed in detail in the “Examples” section, the model described herein (which integrates input data of images and non-imaging clinical data) obtained AUC of 0.91±0.002 for cancer-positive biopsy prediction, and 0.85±0.001 on the identification of “normal” exams. On the sub-cohort where radiologists' final BI-RADS could be estimated on DM alone, without the assistance of US, the performance was even better than in the general cohort (AUC of 0.94±0.002 vs. 0.91±0.002).

In comparison to existing clinical-based risk models (e.g., as described with reference to Meads C, Ahmed I, Riley R D. A systematic review of breast cancer incidence risk prediction models with meta-analysis of their performance. Breast cancer research and treatment. 2012; 132 (2):365-377. doi:10.1007/s10549-011-1818-2), the prediction on clinical data alone by the classifier component of the model outperformed Gail's (p<0.004) (e.g., as described with reference to Gail M H, Brinton L A, Byar D P, et al. Projecting Individualized Probabilities of Developing Breast Cancer for White Females Who Are Being Examined Annually. JNCI Journal of the National Cancer Institute. 1989; 81 (24):1879-1886. doi:10.1093/jnci/81.24.1879). As deep learning (DL) algorithms often lack interpretability, combining DL with non-imaging clinical data may shed a light on their results. For example, first, by offering a careful cohort selection and avoidance/adjustment for biases. Second, by using clinically-centered features, physicians may be able to transcend correlations-based predictions into causal networks of clinical factors leading to a diagnosis.

At least some implementations of the systems, methods, apparatus, and/or code instructions described herein provide improvements over prior approaches. Previous studies applied machine learning and deep learning of breast cancer to relatively small sets, typically less than 2500 individuals, based on subsets of the DDSM dataset, INBreast or BCDR datasets, for example, as described with reference to Heath M, Bowyer K, Kopans D, Moore R, Kegelmeyer W P. The digital database for screening mammography. In: Medical Physics Publishing; 2000:212-218, Moreira I C, Amaral I, Domingues I, Cardoso A, Cardoso M J, Cardoso J S. Inbreast: toward a full-field digital mammographic database. Academic radiology. 2012; 19 (2):236-248, Moura D C, López M A G, Cunha P, et al. Benchmarking Datasets for Breast Cancer Computer-Aided Diagnosis (CADx). In: Ruiz-Shulcloper J, Sanniti di Baja G, eds. Progress in Pattern Recognition, Image Analysis, Computer Vision, and Applications. Vol 8258. Berlin, Heidelberg: Springer Berlin Heidelberg; 2013:326-333. doi:10.1007/978-3-642-41822-8_41. Some studies report results on full images, for example, as described with reference to Carneiro G, Nascimento J, Bradley A P. Unregistered multiview mammogram analysis with pre-trained deep learning models. In: Springer; 2015:652-660, Dhungel N, Carneiro G, Bradley A P. Fully automated classification of mammograms using deep residual neural networks. In: IEEE; 2017:310-314, Zhu W, Lou Q, Vang YS, Xie X. Deep multi-instance networks with sparse label assignment for whole mammogram classification. In: Springer; 2017:603-611, while others focus on region of interest patch classification, for example, as described with reference to Kooi T, Litjens G, van Ginneken B, et al. Large scale deep learning for computer aided detection of mammographic lesions. Medical image analysis. 2017; 35:303-312, Arevalo J, González F A, Ramos-Pollán R, Oliveira J L, Lopez M A G. Representation learning for mammography mass lesion classification with convolutional neural networks. Computer methods and programs in biomedicine. 2016; 127:248-257, Becker A S, Marcon M, Ghafoor S, Wurnig M C, Frauenfelder T, Boss A. Deep Learning in Mammography: Diagnostic Accuracy of a Multipurpose Image Analysis Software in the Detection of Breast Cancer. Investigative Radiology. 2017; 52 (7):434-440. doi:10.1097/RLI.0000000000000358. The DM DREAM Challenge, for example, as described with reference to D.R.E.A.M. The digital mammography DREAM challenge. 2017. https://www(dot)synapse(dot)org/Digital_Mammography_DREAM_challenge. provided the largest existing DM data set confirmed with tissue diagnosis, consisting of 86K individuals. Their task was to develop an automatic algorithm for breast cancer screening classification, where only global information of biopsy-positive labels was provided, first without clinical information and then with a limited set of features. The winning team obtained an AUC of 0.87, and specificity 81% at sensitivity 80%. Another large study used 103K images from 23K exams and focused on breast cancer screening with BI-RADS classes 0, 1 or 2, corresponding to exams that are incomplete, normal, or with benign findings, as described with reference to Geras K J, Wolfson S, Shen Y, Kim S, Moy L, Cho K. High-resolution breast cancer screening with multi-view deep convolutional neural networks. arXiv preprint arXiv:170307047. 2017. Risk prediction models may also improve, by considering genetic information, hormone measurements, and breast density, for example, as described with reference to Howell A, Anderson A S, Clarke R B, et al. Risk determination and prevention of breast cancer. Breast Cancer Research. 2014; 16 (5):446. doi:10.1186/s13058-014-0446-2. Some studies have shown improvement by adding breast density, for example, as described with reference to Tice J A, Cummings S R, Ziv E, Kerlikowske K. Mammographic breast density and the Gail model for breast cancer risk prediction in a screening population. Breast Cancer Res Treat. 2005; 94 (2):115-122. doi:10.1007/s10549-005-5152-4, Chen J, Pee D, Ayyagari R, et al. Projecting Absolute Invasive Breast Cancer Risk in White Women With a Model That Includes Mammographic Density. JNCI: Journal of the National Cancer Institute. 2006; 98 (17):1215-1226. doi:10.1093/jnci/djj332. Wu et al. as described with reference to Wu Y, Abbey C K, Chen X, et al. Developing a utility decision framework to evaluate predictive models in breast cancer risk estimation. Journal of Medical Imaging. 2015; 2 (4):041005. doi:10.1117/1.JMI.2.4.041005 used Gail et al.'s features with and without mined mammographic features, using a logistic regression-based model resulted in AUC of 0.71 vs. 0.60, respectively.

Previous attempts at incorporation some clinical features with imaging data relied on a manual radiologist analysis and interpretation of the current DM image. In contrast, the architecture of the model described herein automatically integrates and analyzes images and non-imaging clinical data based on the intermediate vector that stores embedding values computed for the anatomical image(s), values outputted by a dense layer of the neural network component in response to an input of at least some of the non-imaging clinical parameters, and an intermediate value indicative of likelihood of malignancy for the target tissue, as described herein.

The model described herein, which is trained on a dataset of images (e.g., mammograms) and non-imaging clinical parameters (e.g., health records) provides improved results over previous risk models and obtained performance in the acceptable range of radiologists for breast cancer screening, as described herein. The model may be trained on, and/or receive as input, for example, genetic information, ultrasound images, and/or prior imaging data, which may further improve the results. The model described herein links datasets from multiple imaging and non-imaging modalities to improve the accuracy of malignancy detection, which may save valuable expert time on high probability healthy individuals. In particular, the ability to lower false negative results by half is of immediate clinical relevance.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

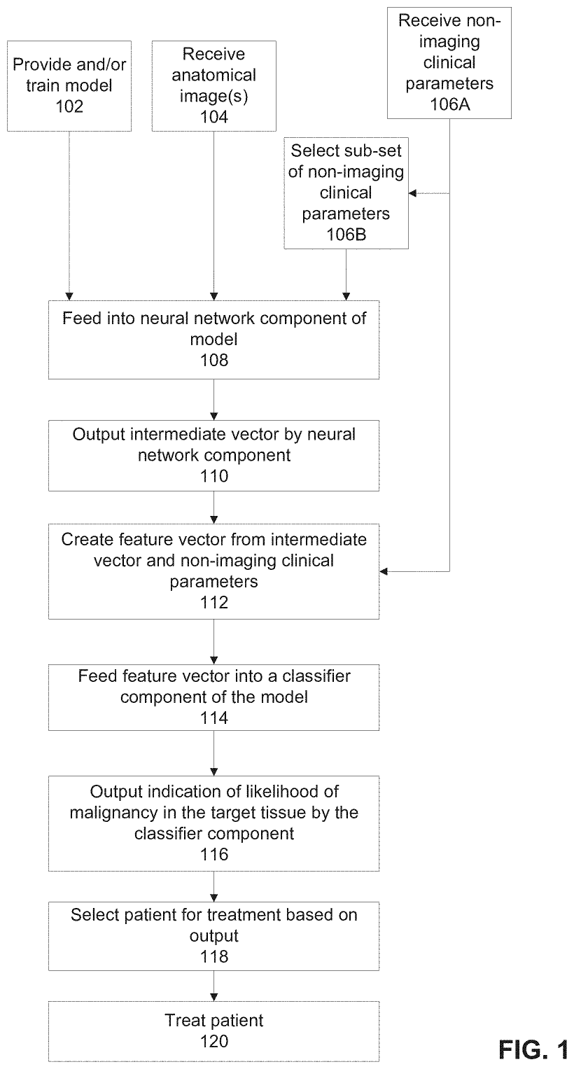

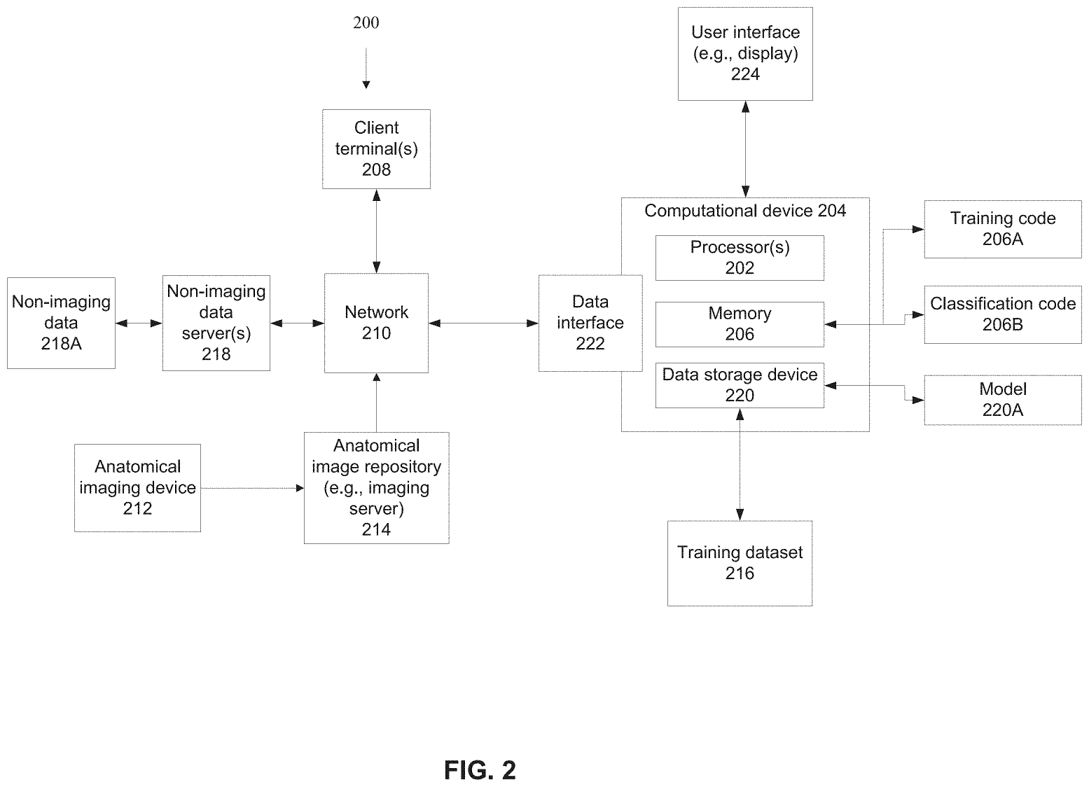

Reference is now made to FIG. 1, which is a flowchart of a method for computing an indication of likelihood of malignancy in a target tissue of a patient by a trained model based on a combination of images and non-imaging clinical data, in accordance with some embodiments of the present invention. The indication of likelihood of malignancy may be used for planning treatment of a patient, for example, determining whether to perform a biopsy of the target tissue. Reference is also made to FIG. 2, which is a block diagram of components of a system 200 for computing an indication of likelihood of malignancy in a target tissue of a patient by a trained model and/or for training the model based on a combination of images and non-imaging clinical data, in accordance with some embodiments of the present invention. Reference is also made to FIG. 3, which is a flowchart of a method of training the model for computing the indication of likelihood of malignancy in the target tissue of the patient based on a combination of images and non-imaging clinical data, in accordance with some embodiments of the present invention. System 200 may implement the features of the method described with reference to FIGS. 1 and/or 3, by one or more hardware processors 202 of a computing device 204 executing code instructions stored in a memory (also referred to as a program store and/or storage device) 206, for example, training code 206A, classification code 206B, and/or model code 220A.

Computing device 204 may be implemented as, for example one or more and/or combination of: a client terminal, a server, a radiology workstation, an imaging server (e.g., PACS), an electronic medical record (EMR) server, a virtual machine, a virtual server, a computing cloud, a mobile device, a desktop computer, a thin client, a Smartphone, a Tablet computer, a laptop computer, a wearable computer, glasses computer, and a watch computer.

Computing device 204 may be implanted as an add-on to clinical software, for example, to a radiology workstation, a PACS server (or other medical imaging storage server), an EMR server, and/or other patient management software.

Computing device 204 may include locally stored software that performs one or more of the acts described with reference to FIG. 1 and/or FIG. 3, and/or may act as one or more servers (e.g., network server, web server, a computing cloud, virtual server) that provides services (e.g., one or more of the acts described with reference to FIG. 1 and/or FIG. 3) to one or more client terminals 208 (e.g., client terminal used by a user for viewing anatomical images, client terminal running EMR access software, client terminal running patient management software, remotely located radiology workstations, remote picture archiving and communication system (PACS) server, remote electronic medical record (EMR) server) over a network 210, for example, providing software as a service (SaaS) to the client terminal(s) 208, providing an application for local download to the client terminal(s) 208, as an add-on to a web browser and/or a medical imaging viewer application and/or EMR viewing application and/or other patient management application, and/or providing functions using a remote access session to the client terminals 208, such as through a web browser, application programming interface (API), and/or software development kit (SDK).

Computing device 204 receives one or more anatomical images (e.g., 2D images, and/or 2D slices of a 3D volume) captured by an anatomical imaging device(s) 212, for example, a standard two dimensional (2D) anatomical imaging device, a sequence of 2D anatomical images (e.g., captured by a fluoroscopic machine), and/or a three dimensional (3D) anatomical imaging device from which 2D images are optionally extracted as slices (e.g., CT, MRI). Anatomical imaging machine(s) 212 may include a standard x-ray based machine, a mammogram machine, a CT scanner, an MRI machine, a colonoscope, an endoscope, and an ultrasound machine.

Anatomical images captured by anatomical imaging machine 212 may be stored in an anatomical imaging repository 214, for example, an imaging storage server, a data storage server, a computing cloud, a PACS server (picture archiving and communication system), and a hard disk. The anatomical images stored by anatomical imaging repository 214 include anatomical images of patients for analysis, and/or images of sample patients included in a training dataset 216 for training the model, as described herein.

Exemplary anatomical images include mammographic images, images captured by a colonoscope, CT scans (e.g., chest CT, abdominal CT), MRI scans, and ultrasound scans.

Computing device 204 receives non-imaging data of the patient 218A, which may be stored in a non-imaging data server(s) 218, for example, an EMR server, a laboratory server storing values of laboratory performed tests (e.g., blood test, genetic test, urinalysis). The non-imaging data of sample patients may be included in training dataset 216 for training the model, as described herein.

Computing device 204 may receive the anatomical image(s) and/or non-imaging data via one or more data interfaces 222, for example, a wire connection (e.g., physical port), a wireless connection (e.g., antenna), a network interface card, other physical interface implementations, and/or virtual interfaces (e.g., software interface, application programming interface (API), software development kit (SDK), virtual network connection).

Hardware processor(s) 202 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 202 may include one or more processors (homogenous or heterogeneous), which may be arranged for parallel processing, as clusters and/or as one or more multi core processing units.

Memory 206 stores code instructions executable by hardware processor(s) 202. Exemplary memories 206 include a random access memory (RAM), read-only memory (ROM), a storage device, non-volatile memory, magnetic media, semiconductor memory devices, hard drive, removable storage, and optical media (e.g., DVD, CD-ROM). For example, memory 206 may store classification code 206B that execute one or more acts of the method described with reference to FIG. 1 and/or training code 206A that execute one or more acts of the method described with reference to FIG. 3.

Computing device 204 may include a data storage device 220 for storing data, for example, a trained model 220A as described herein, and/or training dataset 216 as described herein. Data storage device 220 may be implemented as, for example, a memory, a local hard-drive, a removable storage unit, an optical disk, a storage device, a virtual memory and/or as a remote server and/or computing cloud (e.g., accessed over network 210). It is noted that model 220A may be stored in data storage device 220, for example, with executing portions loaded into memory 206 for execution by processor(s) 202.

Computing device 204 may connect using network 210 (or another communication channel, such as through a direct link (e.g., cable, wireless) and/or indirect link (e.g., via an intermediary computing unit such as a server, and/or via a storage device) with one or more of:
Client terminal(s) 208, for example, when computing device 204 acts as a server providing services (e.g., SaaS) to remote radiology terminals and/or remote medical servers and/or other remove devices, by analyzing remotely obtained anatomical images and remotely obtained non-imaging data, for computing the likelihood of malignancy in the target tissue.
Anatomical image repository (e.g., imaging server) 214, for example, to obtain the anatomical image(s) of the patient for analysis, and/or to obtain anatomical image(s) of sample patients for inclusion in the training dataset for training the model.
Non-imaging data server(s) 218, for example, to obtain the non-imaging data of the patient for analysis, and/or to obtain non-imaging data of sample patients for inclusion in the training dataset for training the model.

Computing device 204 and/or client terminal(s) 208 include and/or are in communication with a user interface(s) 224 that includes a mechanism designed for a user to enter data (e.g., select patient anatomical images and/or non-imaging data) and/or view the computed indication of malignancy. Exemplary user interfaces 224 include, for example, one or more of, a touchscreen, a display, a keyboard, a mouse, and voice activated software using speakers and microphone.

Referring now back to FIG. 1, at 102, a model for outputting an indication of likelihood of malignancy in a target tissue in response to an input of a combination of anatomical image(s) and/or non-imaging clinical parameters is provided and/or trained, for example, as described with reference to FIG. 3.

Different models may be provided, for example, per type of anatomical imaging modality (e.g., CT, x-ray, MRI, nuclear medicine scan, PET, ultrasound), and/or per target tissue (e.g., breast, prostate, colon, esophagus, liver, pancreas, brain, lung)

At 104, one or more anatomical images are received, for example, from a PACS server.

The anatomical images are of a patient, depicting a target tissue. The model outputs likelihood of malignancy in the target tissue.

Optionally, multiple anatomical images are received. Optionally, the multiple anatomical images are captured by a single imaging modality device of a single imaging modality, optionally during a single imaging session, for example, images of an ultrasound scan, x-ray images, CT scan, MRI scan, nuclear imaging scan, and PET scan. Optionally, each of the anatomical images depicts a respective unique image planes of the target tissue. Different images depict the target tissue at different viewing angles (e.g., AP, PA, lateral, other angles) and/or different imaging planes (e.g., different axial slices) and/or different depths (e.g., for ultrasound images).

At 106A, multiple non-imaging clinical parameters are received, for example, from the EMR of the patient. The non-imaging clinical parameters may be, for example, text values, numerical values, ranges of values, and/or a binary value.

Exemplary categories of non-imaging clinical parameters include: demographics, symptoms, history of the present illness, family history, gynecological history, medications, previous radiological history, blood tests, urinalysis, illegal drug use history, smoking history, occupational history, and history of exposure to carcinogenic substances.

Optionally, the non-imaging clinical parameters exclude an external manual and/or automatic analysis of the anatomical image(s). Alternatively, the non-imaging clinical parameters include an external manual and/or automatic analysis of the anatomical image(s), for example, a manual radiological evaluation performed by a radiologist, for example, a BIRADS assessment score.

At 106B, at least some of the non-imaging clinical parameters may be selected. The selection may be performed according to a statistical correlation between each non-imaging clinical parameter and a ground truth indicative of a diagnosis of malignancy. Optionally, non-imaging clinical parameters above a threshold denoting statistically significant association with malignancy may be selected. The ground truth may be obtained from a training dataset of sample patients.

The selection may be performed by a trained classifier, for example, based on an implementation of a gradient boosting machine (GBM), logistic regression, support vector machine (SVM), neural network, or other architecture.

The selection of at least some of the non-imaging clinical parameters may be performed according to the target tissue and/or indication outputted by the model, for example, likelihood of malignancy in breast tissue, likelihood of malignancy in prostate, likelihood of malignancy in lungs, likelihood of malignancy in brain, likelihood of malignancy in pancreas, and likelihood of malignancy in likelihood of malignancy in liver.

For example, when the model outputs, likelihood of malignancy in breast tissue, exemplary non-imaging clinical parameters include one or more of: demographics, age, last body mass index, maximum body mass index, last body mass index class, maximum body mass index class, gynecological history, age at first menstruation, age at last menstruation, indication of postmenopausal, number of menstruation years, pregnancies count, past pregnancies, indication of is breastfeeding, number of children breastfed, indication of current use of hormone replacement therapy, cancer history, family breast cancer first degree, family breast or ovarian cancer, number of relatives with breast or ovarian cancer, minimum age in family for cancer, any personal cancer history, symptoms, lump complaint by woman, bilateral lump complaint by woman, lump complaint by woman in the past, pain complaint by woman, bilateral pain complaint by woman, breast radiology history, past number of breast imaging encounters, past breast density, past final BIRADS assessment DM left, past final BIRADS assessment DM right, past final BIRADS assessment US left, past final BIRADS assessment US right.

At 108, the anatomical image(s), and the non-imaging clinical parameters of the patient are fed into the neural network component of a model.

Optionally, the neural network component include a first sub-neural network component and second sub-neural network component. The anatomical image(s) and the non-imaging clinical parameters are fed into the first sub-neural network component. The anatomical image(s) is fed into the second sub-neural network component. None of the non-imaging clinical parameters are fed into the second sub-neural network component.

Alternatively or additionally, the neural network includes an ensemble of neural networks, each differing by one or more neural network parameter, for example, variation in weights, number of layers, and/or variation in architecture of one or more layers (e.g., connectivity, number of neurons).

At 110, the neural network component outputs an intermediate vector. The intermediate vector includes the following values outputted by the neural network component: embedding values computed for the anatomical image, values outputted by a dense layer of the neural network component in response to an input of at least some of the non-imaging clinical parameters, and an intermediate value indicative of likelihood of malignancy for the target tissue. The intermediate value may be outputted by an output layer of the neural network component that is fed the anatomical image(s) and the non-imaging clinical parameters.

Optionally, when multiple anatomical images are fed into the neural network component, outputs of neural network component generated for each one of the anatomical images are aggregated to compute the intermediate vector.

Optionally, when the neural network component is implemented as an ensemble of neural networks and multiple anatomical images are fed into each neural network of the ensemble, the intermediate vector is computed as an aggregation of the embedding values computed by the ensemble of neural networks, and an aggregation of values outputted by each respective dense layer of the ensemble of neural networks.

Optionally, the intermediate vector is computed as an output of a last fully connected layer that receives a concatenation of an output of a sub-component of the neural network component that is fed the anatomical image, and an output of the dense layer of the neural network component that is fed at least some of the non-imaging clinical parameters. The last fully connected layer outputs the intermediate value indicative of likelihood of malignancy for the target tissue. The sub-component of the neural network component that is fed the anatomical image(s) outputs the embedding values computed for the anatomical image. The neural network component that is fed at least some of the non-imaging clinical parameters outputs the values outputted by the dense layer of the neural network component in response to an input of at least some of the non-imaging clinical parameters.

Optionally, in the implementation where the neural network component include a first sub-neural network component and second sub-neural network component, the intermediate vector includes outputs of the first and second sub-neural network components. The first sub-neural network component outputs the embedding values, the values outputted by the dense layer, and the intermediate value. The second sub-neural network component outputs a second set of embedding values computed for the anatomical image(s) and a second intermediate value indicative of likelihood of malignancy for the target tissue.

At 112, a feature vector may be created from the intermediate vector and the non-imaging clinical parameters.

Optionally, for the implementation of the model where multiple anatomical images each fed into the neural network component, the feature vector may also include metadata values computed for pairs of the anatomical images. The metadata values are computed according to relationships between respective values for the likelihood of malignancy that are computed by the neural network component for the respective pair of images. Exemplary relationships that are computed for a first and second image of each pair include: likelihood of malignancy for the first image divided by a sum of likelihood of malignancy for the first image and likelihood of malignancy for the second image, absolute value of a difference between likelihood of malignancy for the first image and likelihood of malignancy for the second image, and maximum of the likelihood of malignancy for the first image and likelihood of malignancy for the second image.

At 114, the feature vector is fed into a classifier component of the model.

At 116, the classifier component of the model outputs an indication of likelihood of malignancy in the target tissue. It is noted that the indication of likelihood of malignancy in the target tissue may be referred to as being outputted by the model.

At 118, patients may be selected for treatment according to the indication of likelihood of malignancy in the target tissue outputted by the model, for example, patients are selected by electronic tagging of their electronic medical recording with an indication of recommendation for treatment, and/or an electronic message (e.g., email, mail, message, audio call) is generated and provided to the physician suggesting further treatment for the patient.

Optionally, patients having a value indicative of likelihood of malignancy above a threshold are selected for treatment. The threshold may be selected, for example, as indicating statistically significant risk of malignancy that requires further evaluation.

At 120, the selected patients are treated. For example, a biopsy of the target tissue is performed to provide histological evidence for malignancy or lack thereof.

Referring now back to FIG. 3, at 302, data is received for sample patients. For each patient, anatomical image(s), non-imaging clinical parameters, and a ground truth indication of malignancy in a certain target tissue is received. The ground truth indication of malignancy may be obtained, for example, from a diagnosis of malignancy stored in the electronic medical record of the sample patient.

Data and/or patients may be clustered for creating a desired model, for example, according to type of target tissue where malignancy is being evaluated, and/or according to anatomical imaging modality type. For example, one cluster stores data for creating a model for determining likelihood of breast cancer, and another cluster stores data for creating a model for determining likelihood of lung cancer.

At 304, optionally, a sub-set of the non-imaging clinical parameters that are statistically significantly correlated with ground truth indication of malignancy are selected. Optionally, each one of the non-imaging clinical parameters is independently evaluated for statistically significant correlation with the ground truth indication of malignancy. The sub-set of non-imaging clinical parameters may be selected according to a requirement of the statistical correlation, for example, non-imaging clinical parameters having correlation values above a threshold are selected. Exemplary thresholds include: about 0.6, or 0.7, or 0.8, or other smaller, intermediate, or larger values.

Optionally, a classifier is trained to perform the selection, for example, based on an implementation of a GBM classifier, or other architecture.

At 306, a training dataset is created for training the neural network component of the model. The training dataset stores, for each of multiple sample patients, the ground truth indication of malignancy, anatomical image(s) depicting the target tissue, and the selected sub-set of the non-imaging clinical parameters.

Multiple training datasets may be created, for example, according to each cluster.

At 308, the neural network component of the model is trained according to the training dataset.

Optionally, the full set of non-imaging clinical parameters is not used to train the neural network component. The neural network component is trained using the selected sub-set of non-imaging clinical parameters. Alternatively or additionally, none of the non-imaging clinical parameters are used to train the neural network component, for example in implementations in which there are two instances of the neural network component, one of the instances of the neural network component is trained without any of the non-imaging clinical parameters, and the other instance of the neural network component is trained with the selected sub-set of the non-imaging clinical parameters.

The neural network component is trained for outputting an intermediate value indicative of likelihood of malignancy for a target tissue of a target patient, in response to an input of one or more anatomical images depicting the target tissue of the target patient, and the selected sub-set of non-imaging clinical parameters of the target patient.

At 310, an intermediate training dataset is created. The intermediate training dataset is created using outputs generated by the trained neural network component.

The intermediate training dataset stores a respective feature vector and corresponding ground truth indication of malignancy for each of the sample patients. Each feature vector is created from a respective intermediate vector and non-imaging clinical parameters for the respective sample individual, optionally the full set of non-imaging clinical parameters. Each respective intermediate vector stores embedding values computed for the anatomical image(s) of the respective sample individual (the embedding values are computed as described herein), and values outputted by a dense layer of the trained neural network component in response to an input of the selected sub-set of non-imaging clinical parameters of the respective sample individual (i.e., of the training dataset), and an intermediate value indicative of likelihood of malignancy for the target tissue of the sample individual outputted by the trained neural network component.

At 312, the classifier component of the model is trained according to the intermediate training dataset.

At 314, the model is created, by assembling the trained neural network component(s) and the trained classifier component.

At 316, the model is provided, for example, stored in a data storage device, forwarded to another server and/or client terminal, and/or provided for further processing.

The model is provided for selecting patients for treatment according to a computed indication of likelihood of malignancy in a target tissue of a target patient outputted by the model, as described herein.

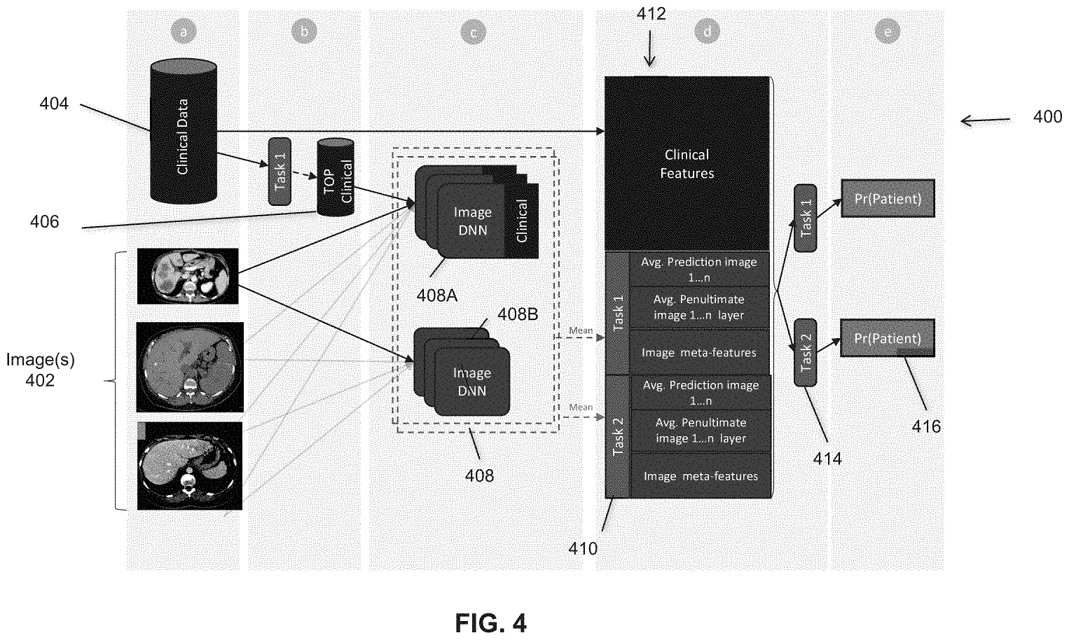

Reference is now made to FIG. 4, which is a schematic depicting an exemplary architecture of a model 400 for outputting an indication of likelihood of malignancy in a target tissue based on a combination of one or more images 402 and non-imaging clinical parameters 404, in accordance with some embodiments of the present invention. As shown, model 400 is designed to output values for Task 1 and Task 2, which are described below in the “Examples” section. One or more images 402 and non-imaging clinical parameters 404 are fed into model 400. A sub-set 406 of non-imaging clinical parameters 404 that are statistically significantly correlated with likelihood of malignancy in the target tissue may be selected. Image(s) 402 and sub-set 406 are fed into a neural network component 408, as described herein.

In the implementation shown, neural network component 408 includes an imaging and non-imaging neural network sub-component 408A, and an imaging only neural network sub-component 408B. Sub-set 406 and images 402 are fed into sub-component 408A. Images 402 are fed into sub-component 408B, i.e., sub-set 406 is not fed into sub-component 408B.

Neural network component 408 outputs one or more intermediate vector 410. It is noted that intermediate vector 410 is marked for Task 2, for example, predicting normal exam versus all other biopsy results (e.g., malignancy, not normal but not necessarily malignant, uncertain results). A similar intermediate vector is outputted for Task 1, for example, predicting a malignant biopsy versus all other exam results (e.g., normal, not normal but not necessarily malignant—biopsy negative, uncertain results). It is noted that more than two tasks may be implemented. It is noted that in the implementation that includes the two tasks, features computed for each task are used for prediction of the other respective tasks, i.e., features of task 1 are provided in the intermediate vector used to compute results for task 2, and features of task 2 are provided in the intermediate vector used to compute results for task 1. In other implementations (i.e., that output a single result), a single intermediate vector is used.

Intermediate vector 410 stores embedding values computed for images 402, values outputted by a dense layer of the neural network component 408 in response to an input of subset 406 of non-imaging clinical parameters, and an intermediate value indicative of likelihood of malignancy for the target tissue.

Feature vector 412 is created from intermediate vector(s) 410 and non-imaging clinical parameters 404.

A classifier component 414 is fed feature vector 412. Classifier component 414 outputs an indication of likelihood of malignancy in the target tissue 416. It is noted that in the implementation for Task 1 and Task 2, two classifiers 414 are used (only one marked for clarity), one classifier for each Task.

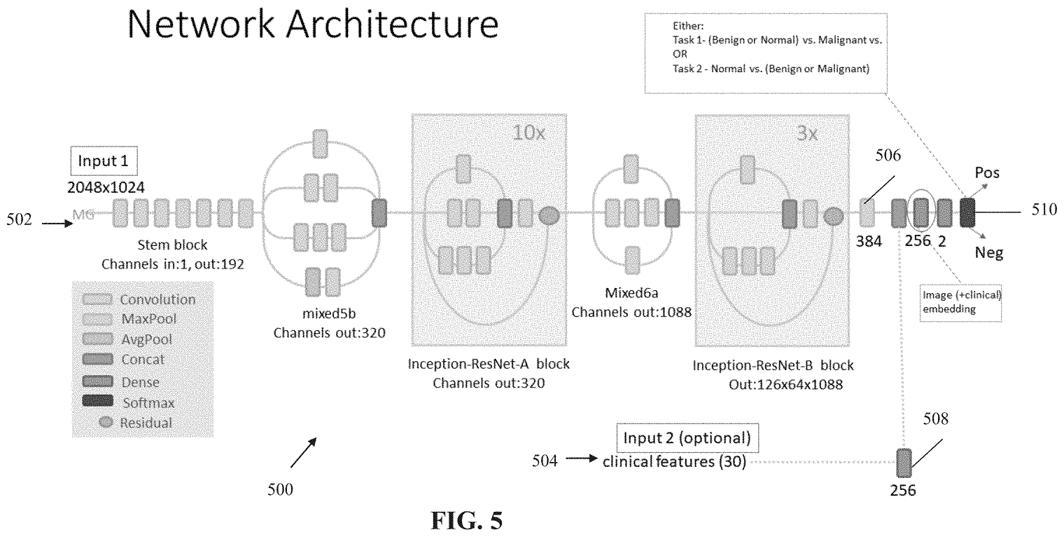

Reference is now made to FIG. 5, which is a block diagram of an exemplary architecture of a neural network component 500 of a model for outputting an indication of likelihood of malignancy in a target tissue based on a combination of one or more images 502 and non-imaging clinical parameters 504, in accordance with some embodiments of the present invention.

Neural network component 500 output an intermediate vector storing embedding values computed for the anatomical image 506, values outputted by a dense layer of the neural network in response to an input of at least some of the non-imaging clinical parameters 508, and an intermediate value indicative of likelihood of malignancy for the target tissue 510. Intermediate value 510 is computed by further processing of a concatenation of embedding values 506 and values of dense layer 508.

Additional details of an exemplary implementation of neural network component 500 are described below in the “Examples” section.

Various embodiments and aspects of the present invention as delineated hereinabove and as claimed in the claims section below find calculated support in the following examples.

### EXAMPLES

Reference is now made to the following examples, which together with the above descriptions illustrate some embodiments of the invention in a non limiting fashion.

Inventors performed a computational evaluation according to the systems and/or methods and/or apparatus and/or code instructions described herein, for example, with reference to FIGS. 1-5, to evaluate the likelihood of malignancy of a target tissue as outputted by the model described herein.

The objectives of the computational evaluation were threefold: to evaluated the ability of the model to predict likelihood of malignancy (i.e., breast cancer) when provided and/or trained on an input of a large dataset of detailed electronic health records (EHR) and DM images; (ii) Identify a set of clinical features fed into the model that demonstrate significant prediction improvement compared to previous risk models and may serve as personalized predictors; (iii) evaluate the results of the model in two key clinical tasks, prediction of cancer-positive biopsy and discrimination of normal from abnormal exams.

Materials and Methods: This retrospective study was approved by the ethics review board of Assuta Medical Centers (AMC), who waived the need to obtain written informed consent. The data were collected and managed by Maccabi Health Services (MHS). MHS and AMC authors (E. H., G. K., V. S., M. G.) obtained the approval for the retrospective study and a confirmation regarding the anonymization process. The analysis was conducted by all other authors who were IBM employees at the time of the study.

Study Design and Patient Eligibility: The cohort comprised women who underwent at least one DM examination between 2013 and 2017 in one of the five AMC imaging facilities, and had at least one year of clinical history in MHS prior to the DM examination. Standard images containing in-breast foreign bodies (clips, markers, pace makers, etc.) of women with previous biopsies (1,848/13,214, 14%) were not excluded. Women with a history of breast cancer, prior breast surgeries (i.e., lumpectomy, breast lift), prior radiotherapy to breast, chemotherapy, implants, and mammograms in which biopsy side was undetermined, were excluded. BI-RADS 1-2 studies without at least two years of normal follow-ups were excluded as well.

For each woman, the first DM examination during the study period that satisfied the inclusion/exclusion criteria was considered the index test. The model used clinical data prior to the index test date. The data in the general cohort was split into three non-overlapping sets: 73% train (9,611 women with 38,444 images), 8% validation (1,055 women with 4,220 images), and 19% test (2,548 women with 10,192 images).

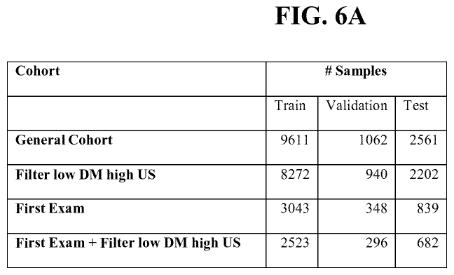

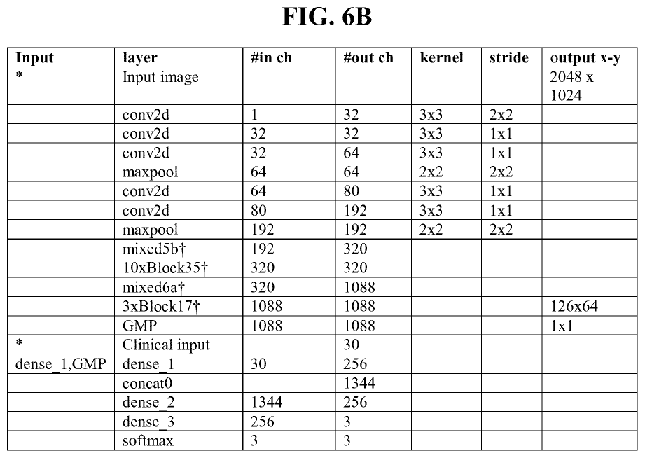

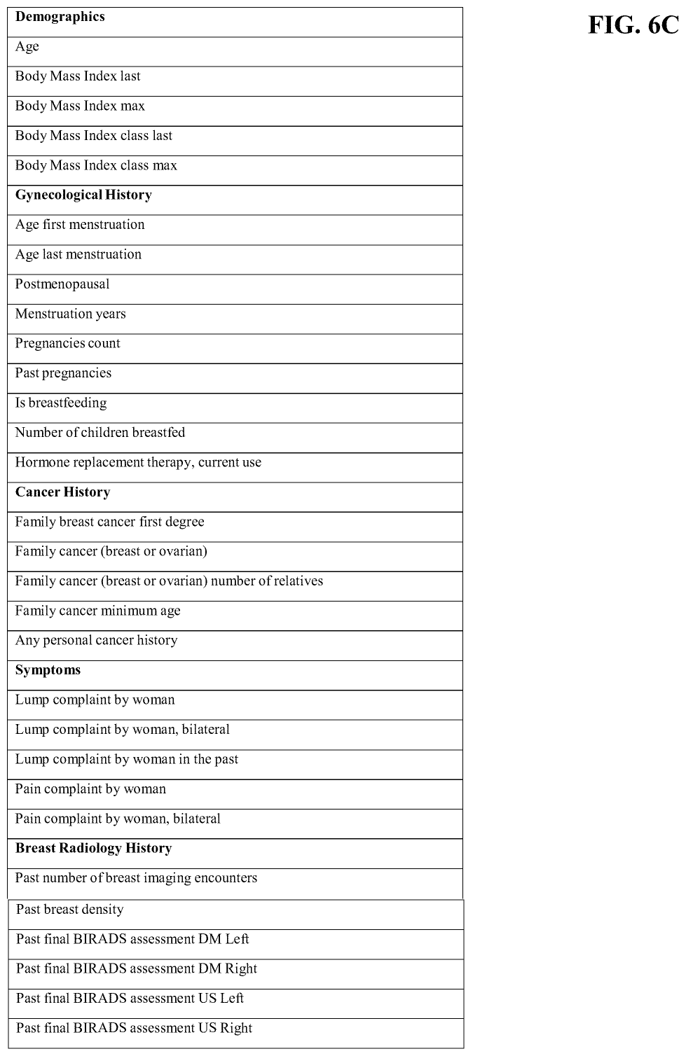

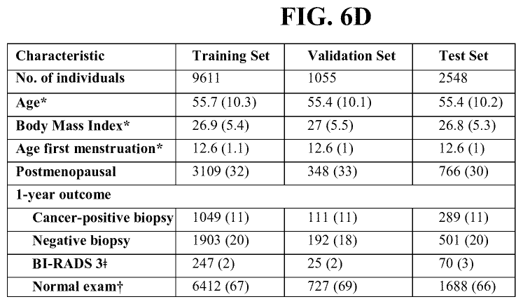

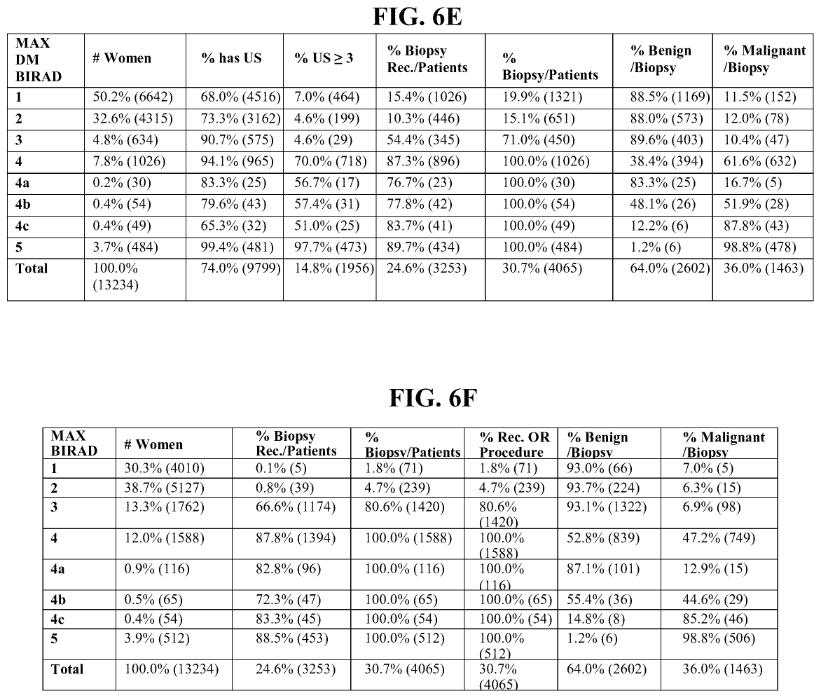

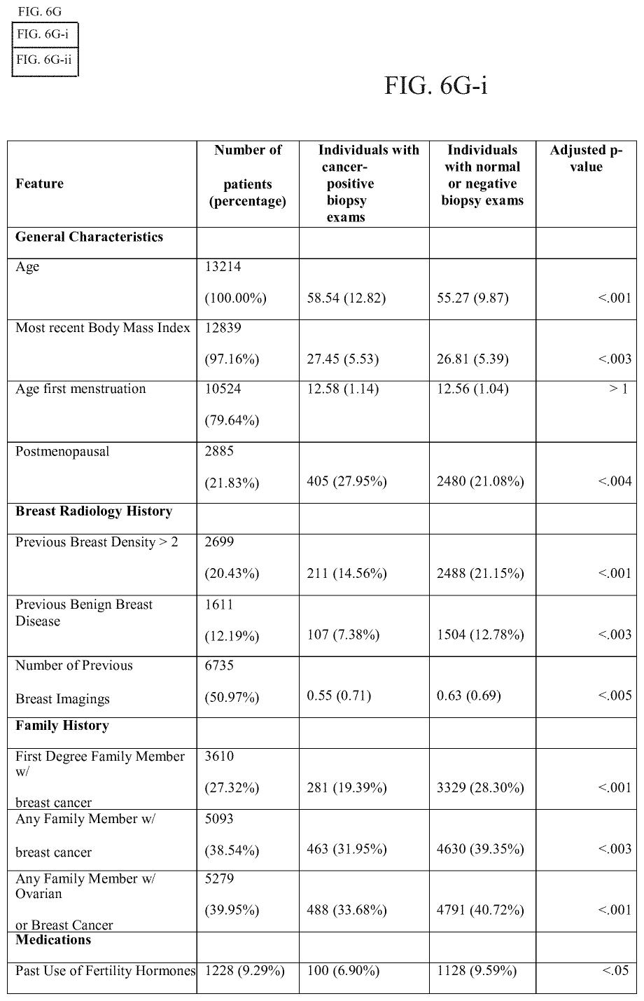

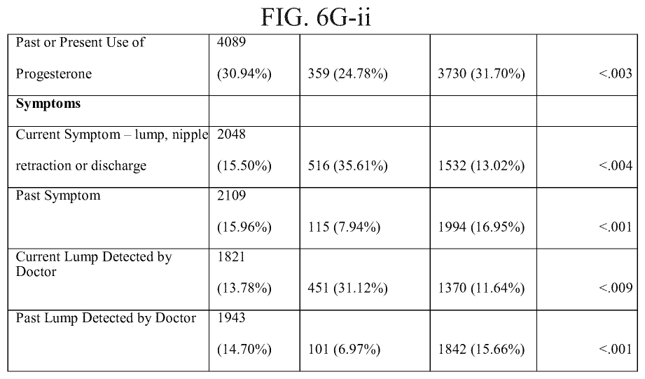

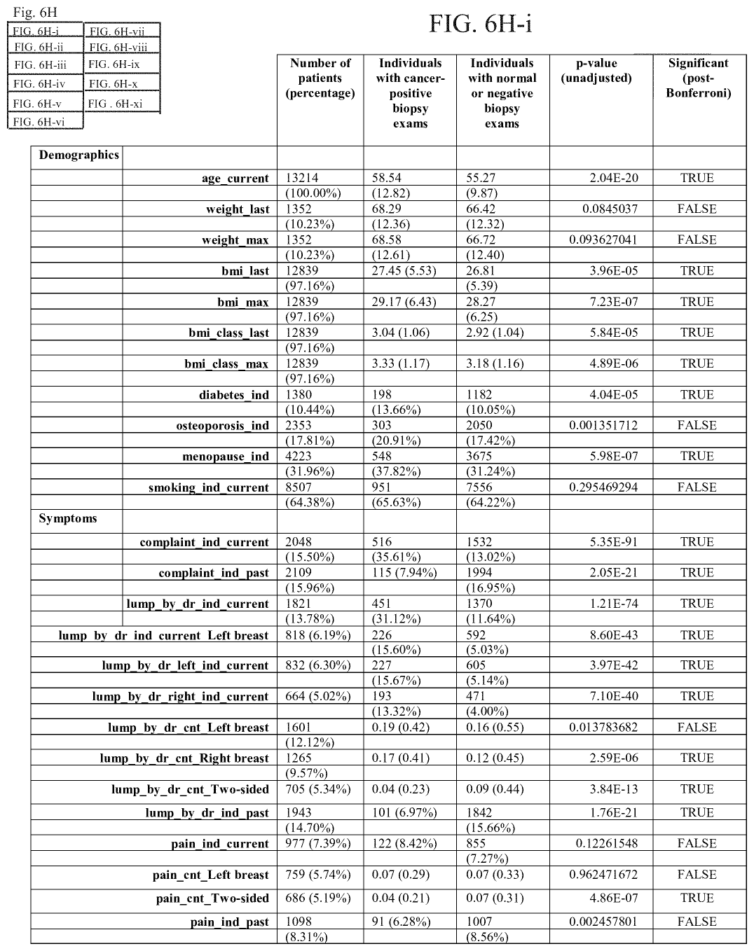

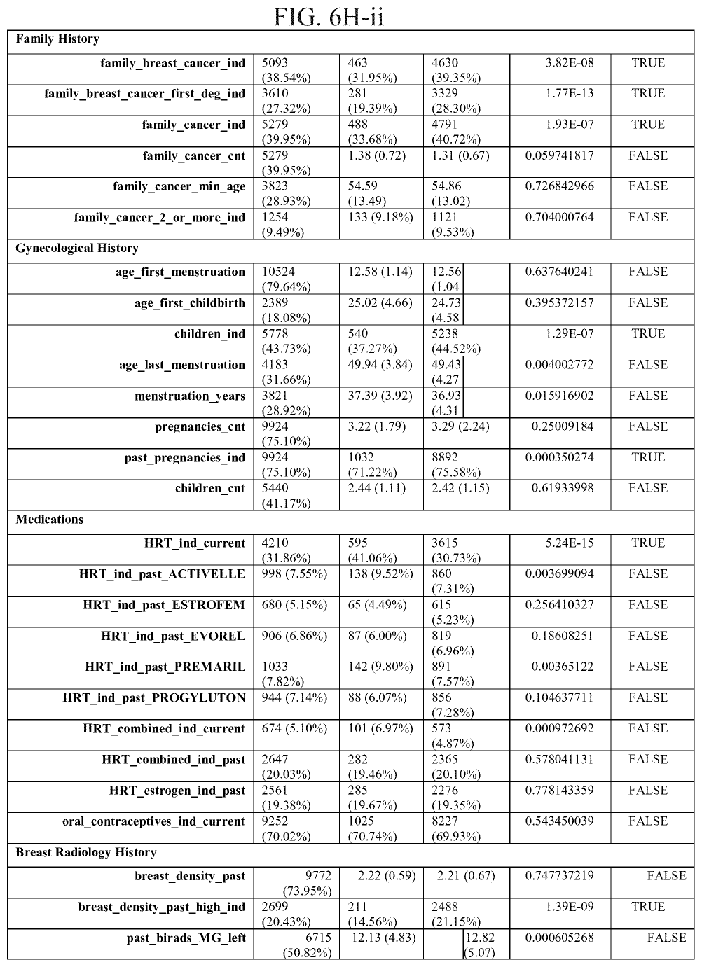

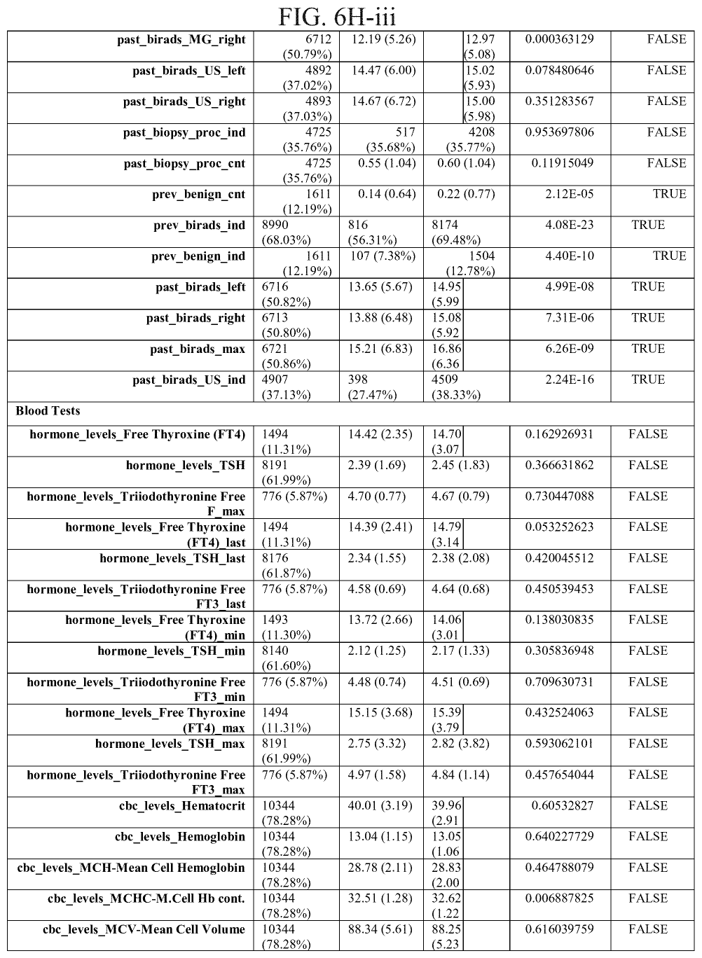

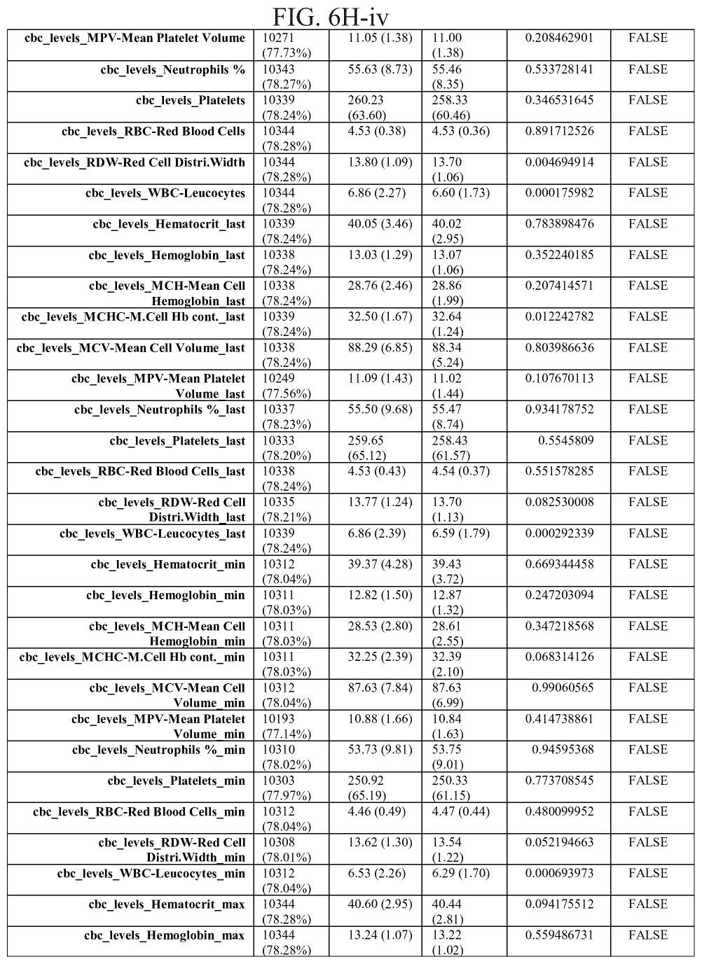

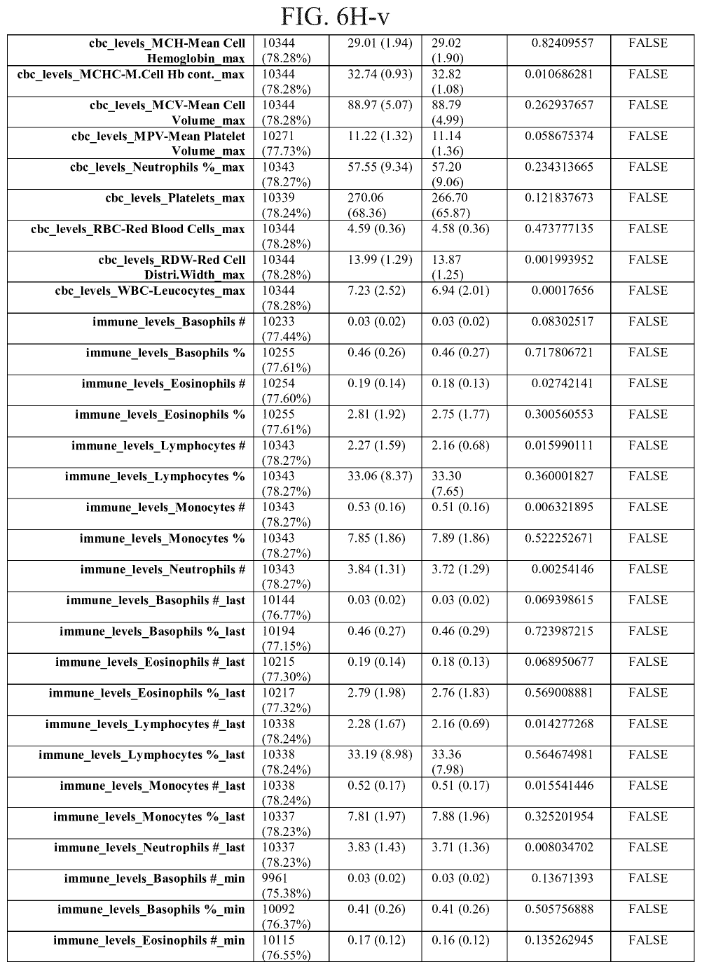

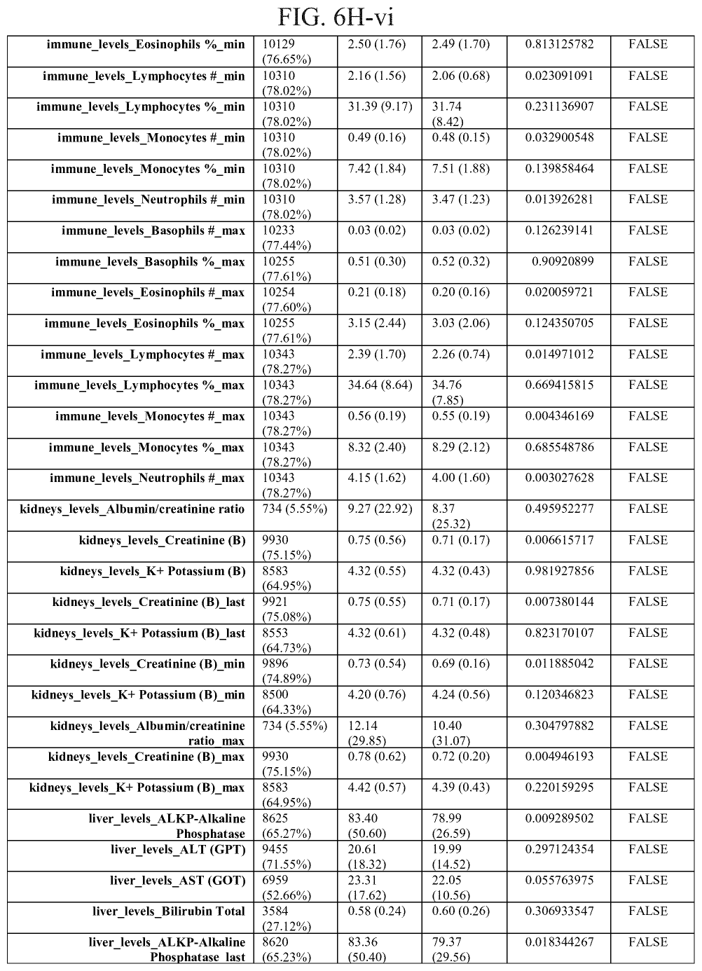

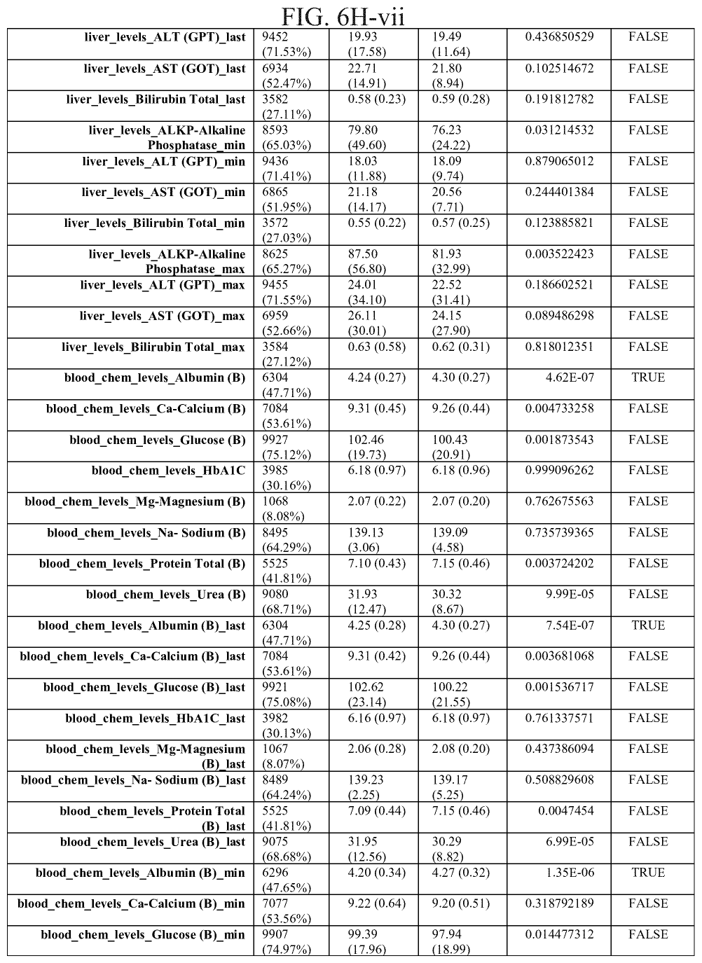

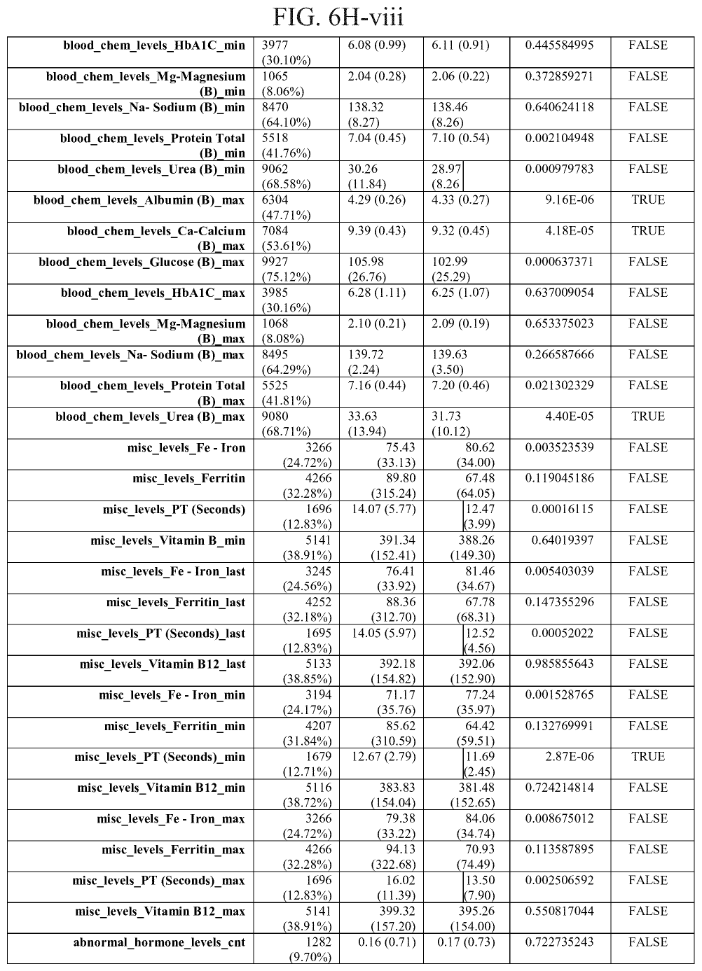

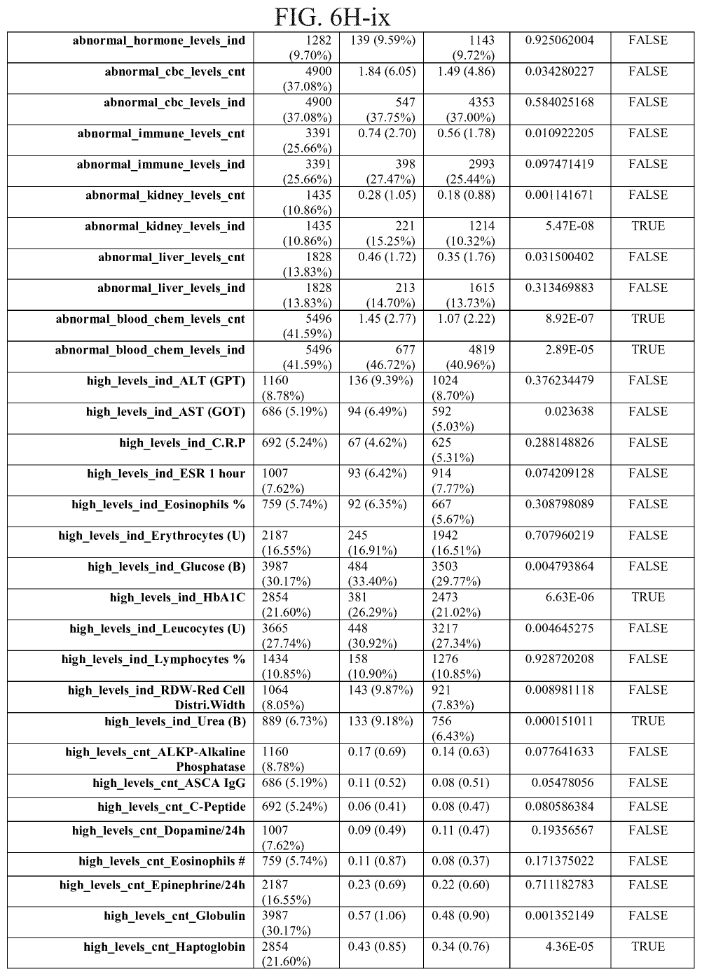

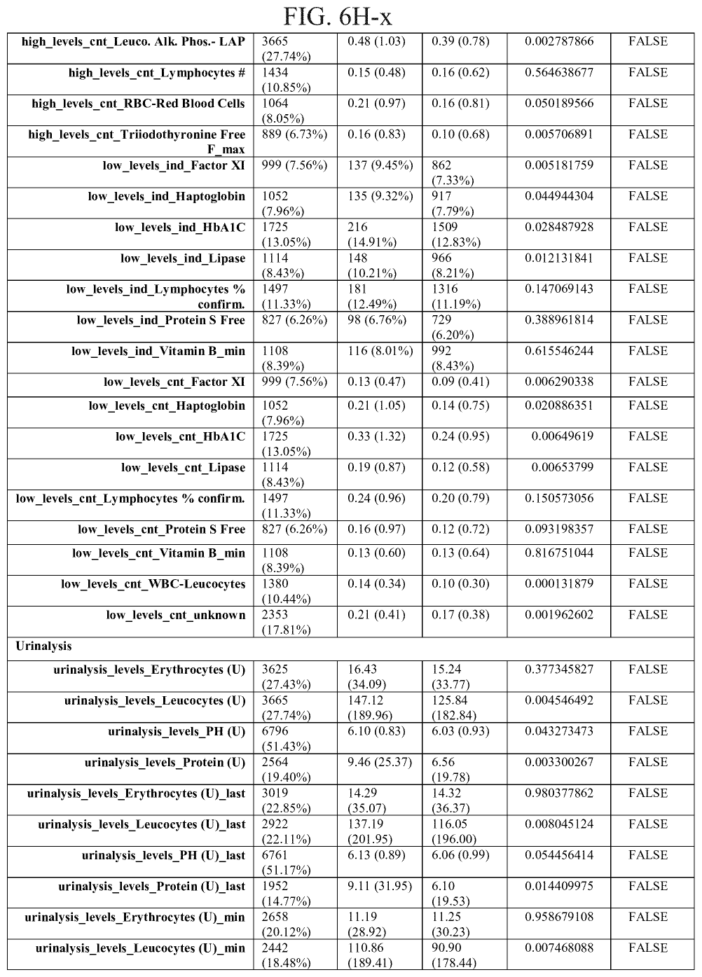

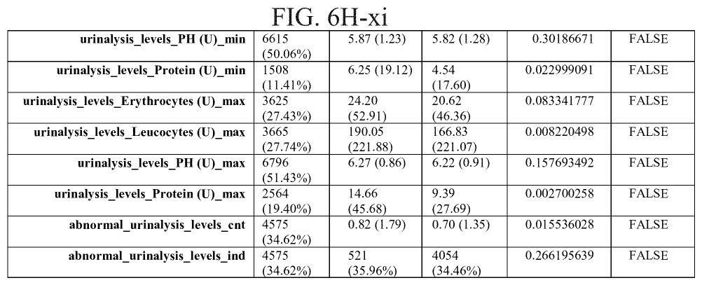

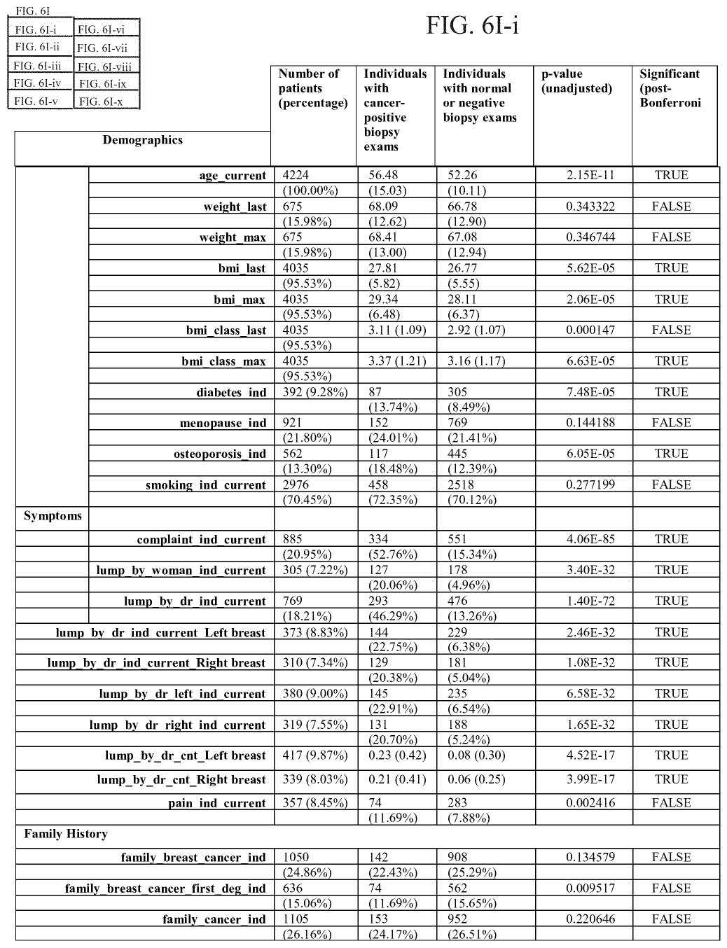

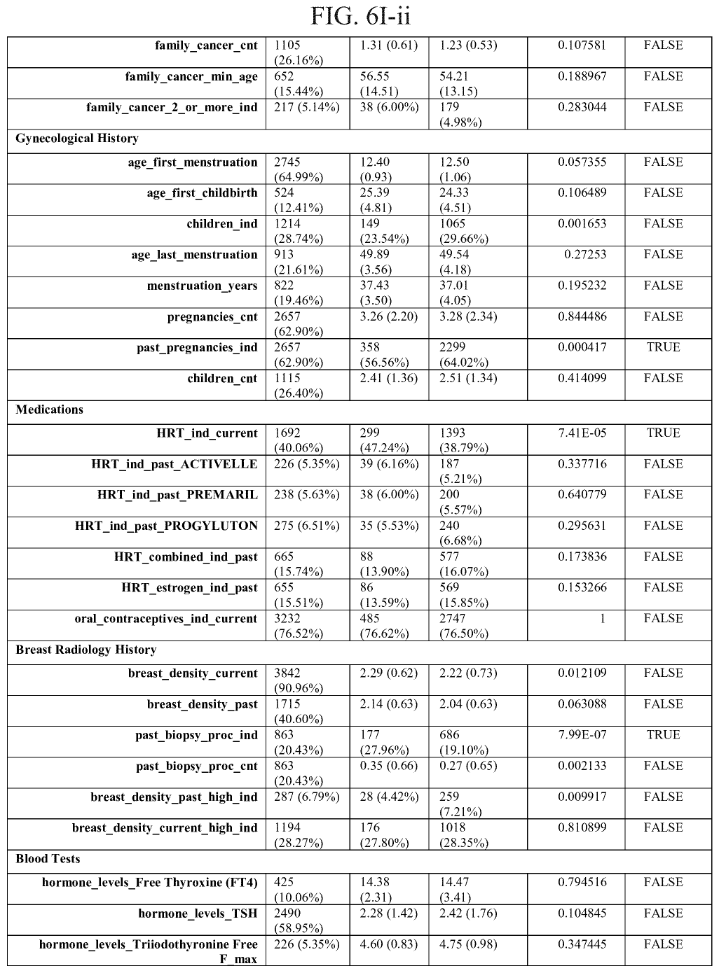

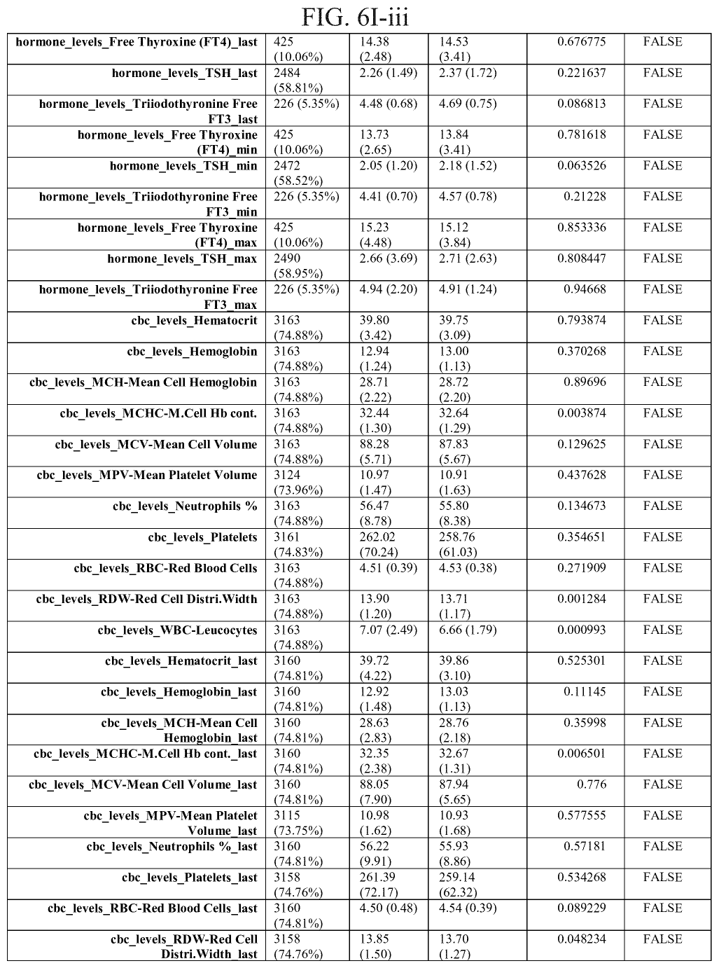

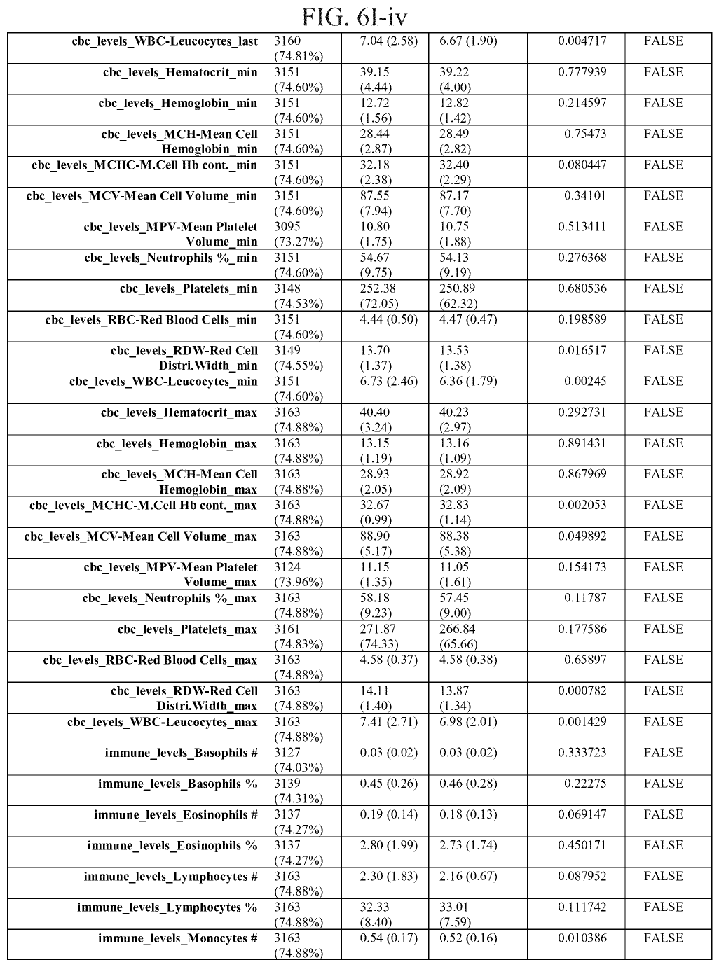

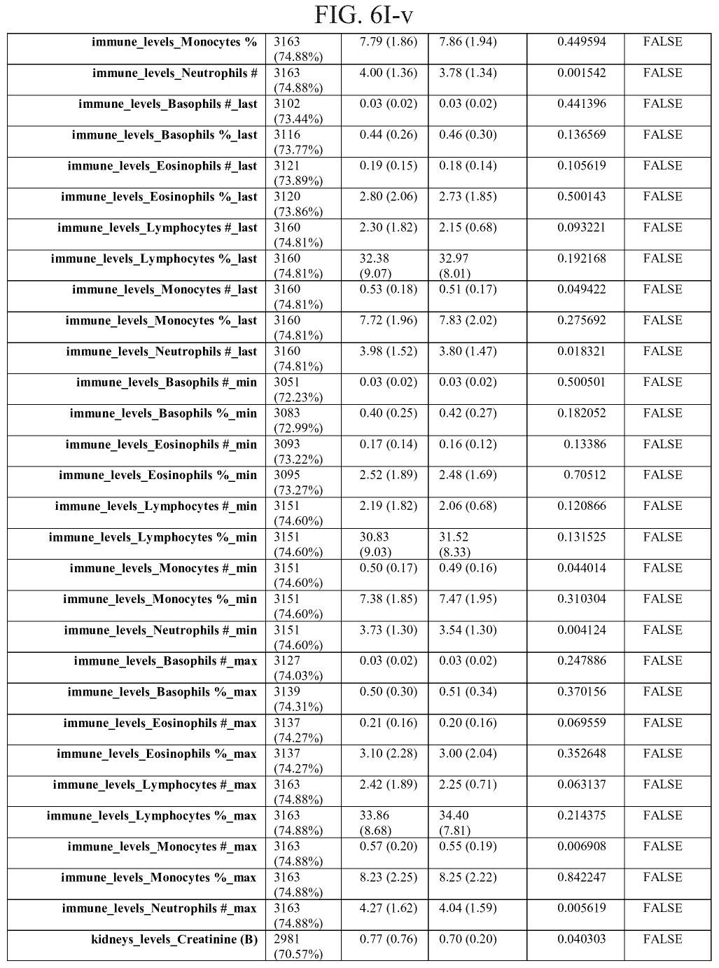

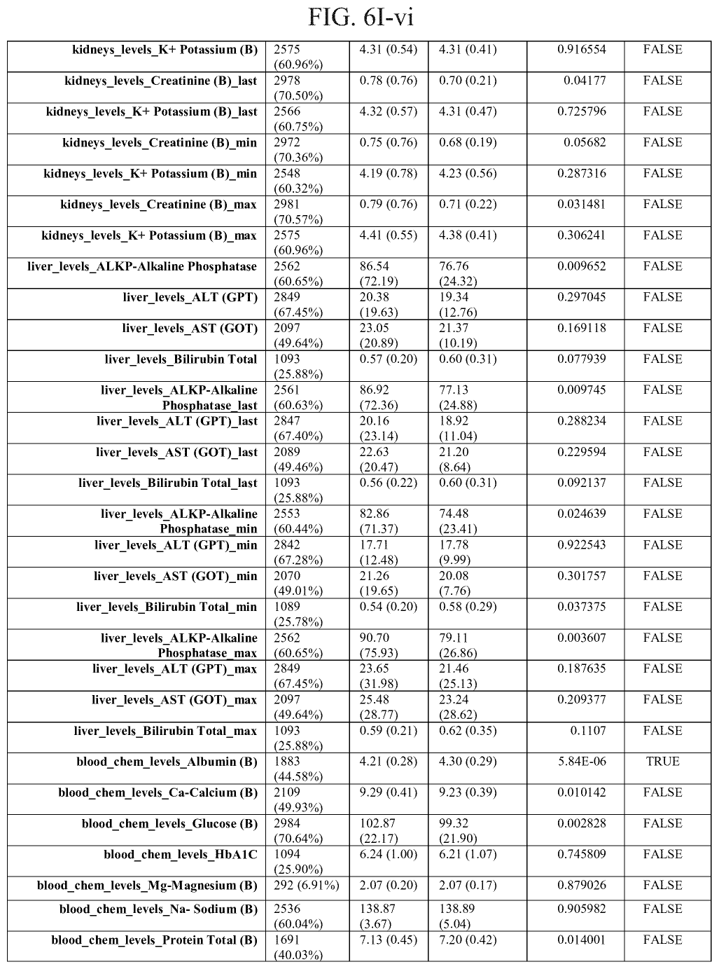

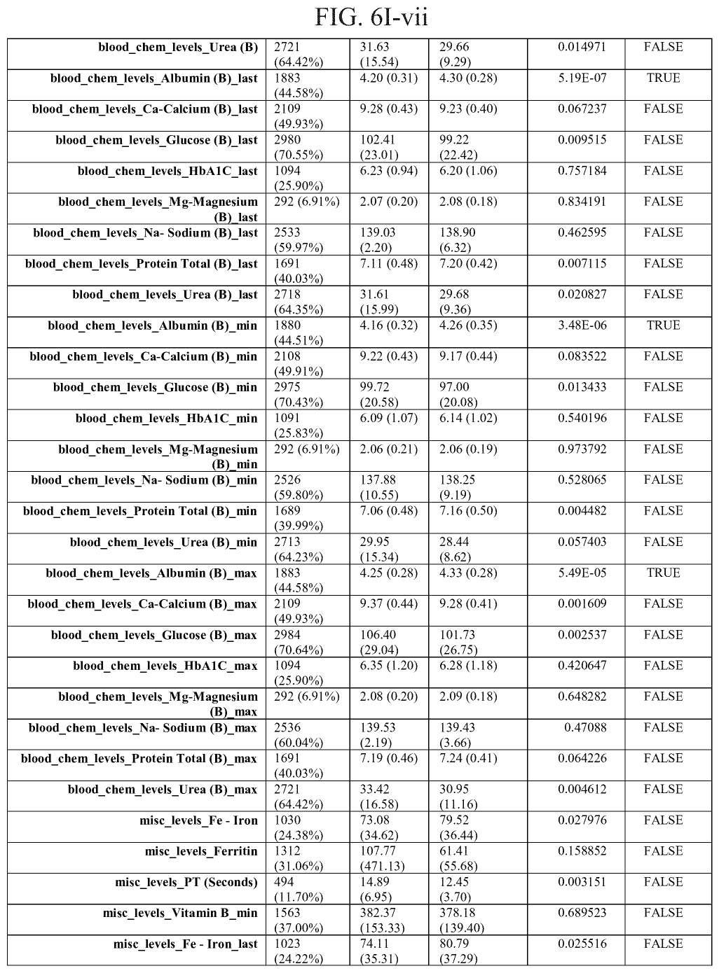

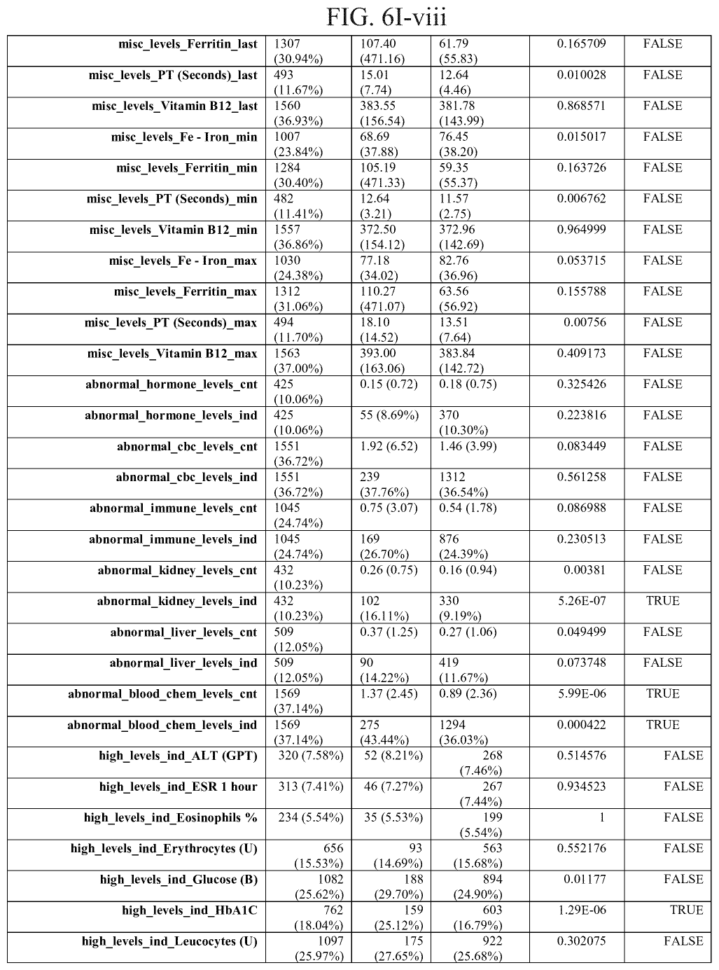

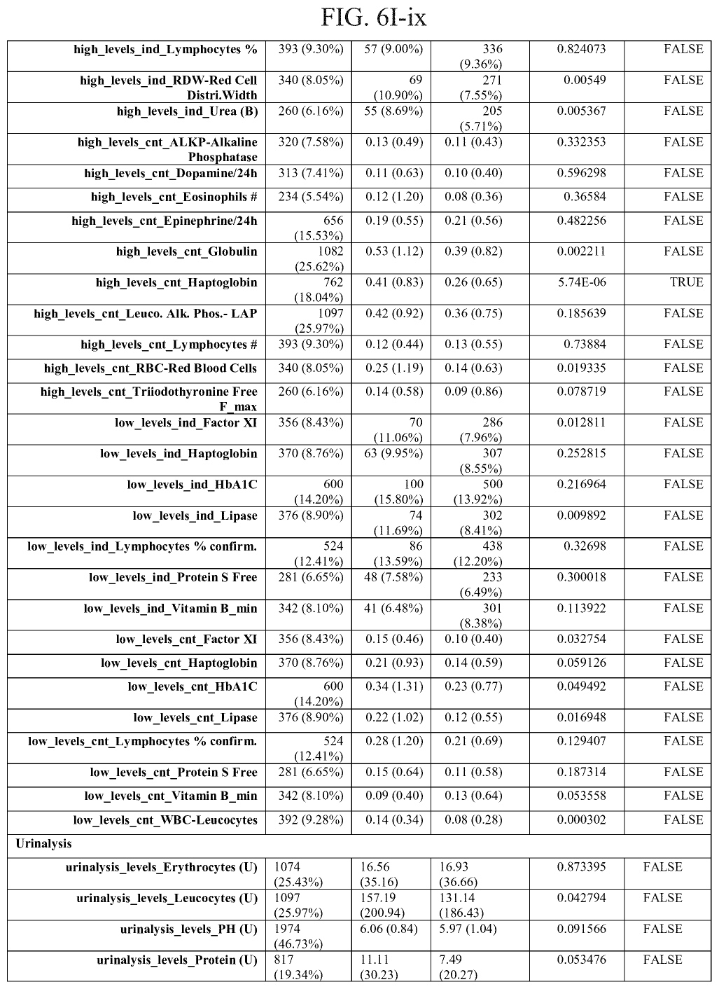

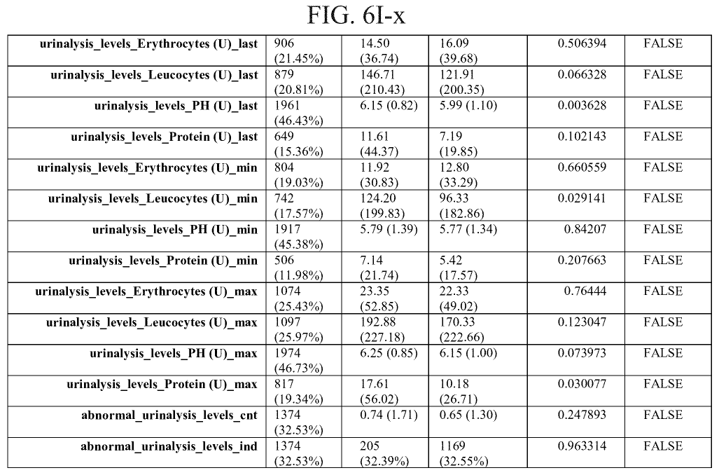

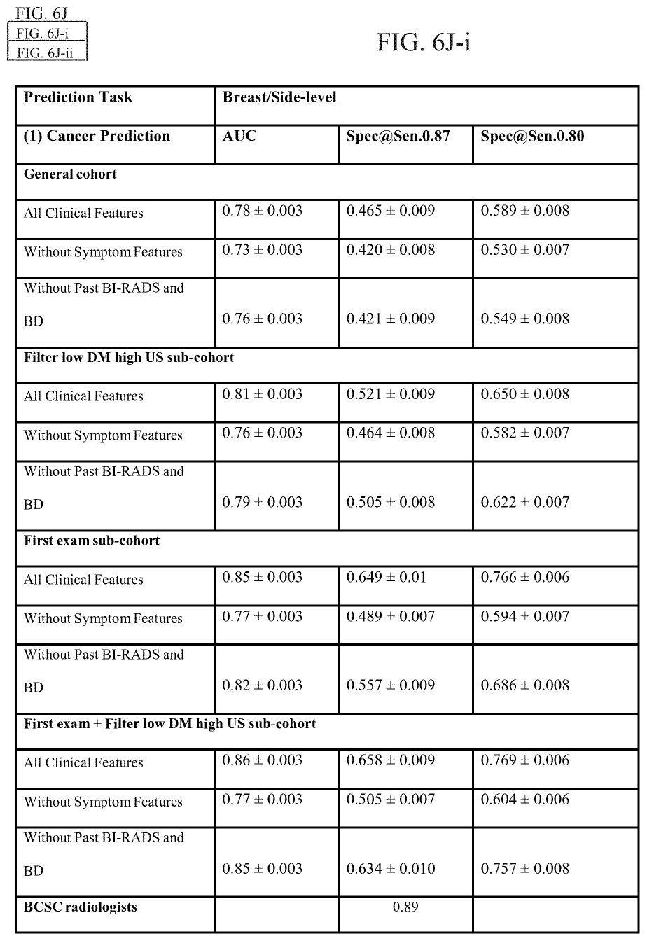

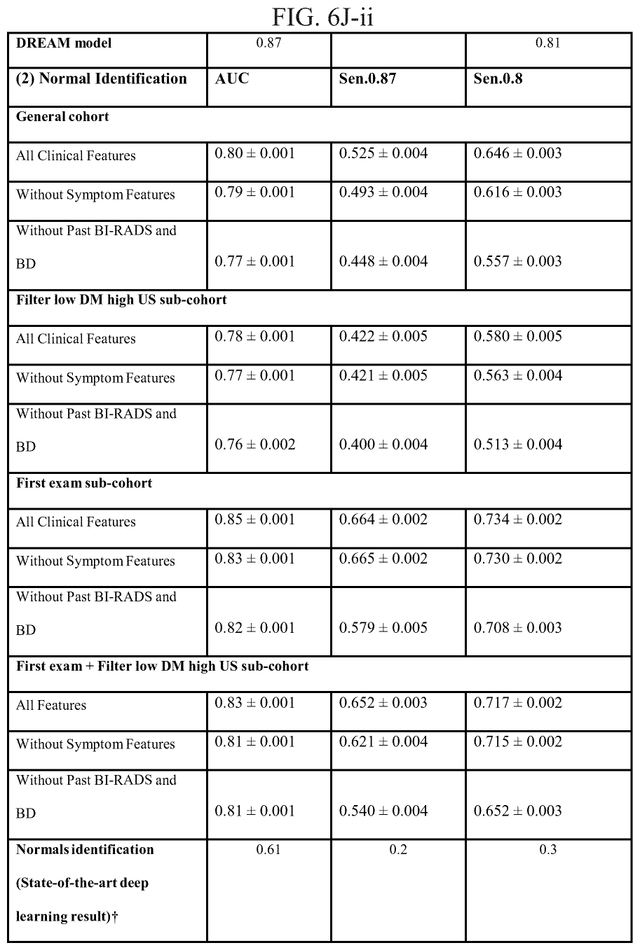

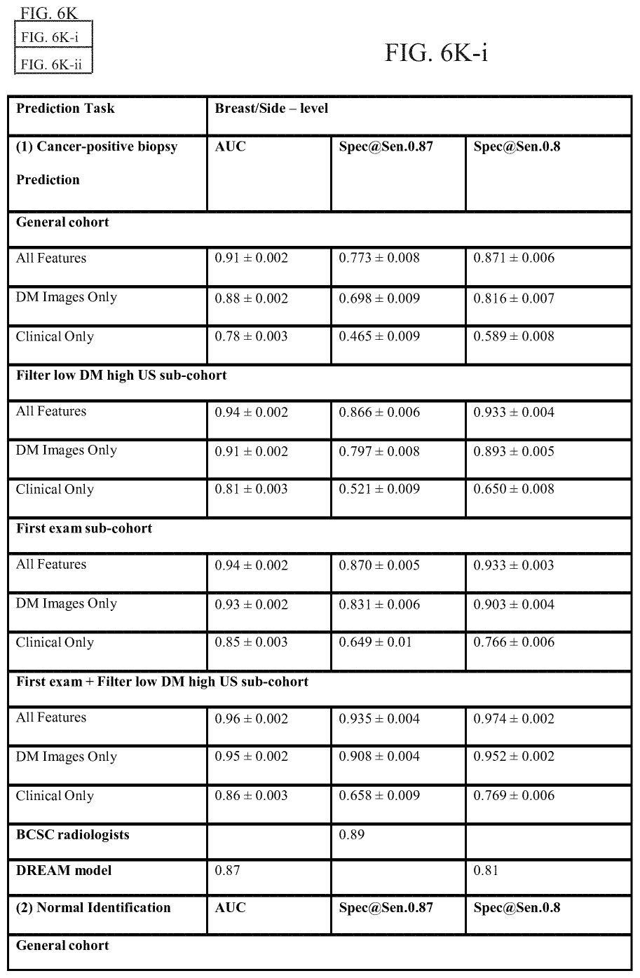

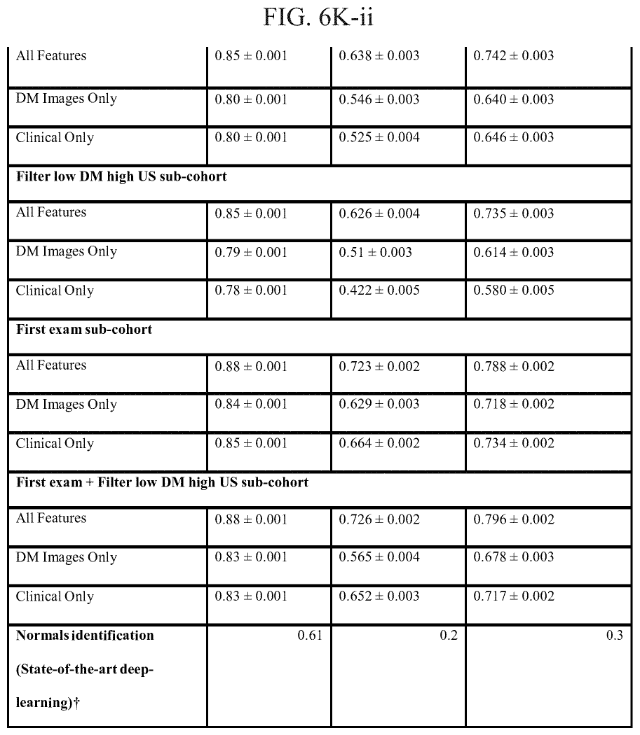

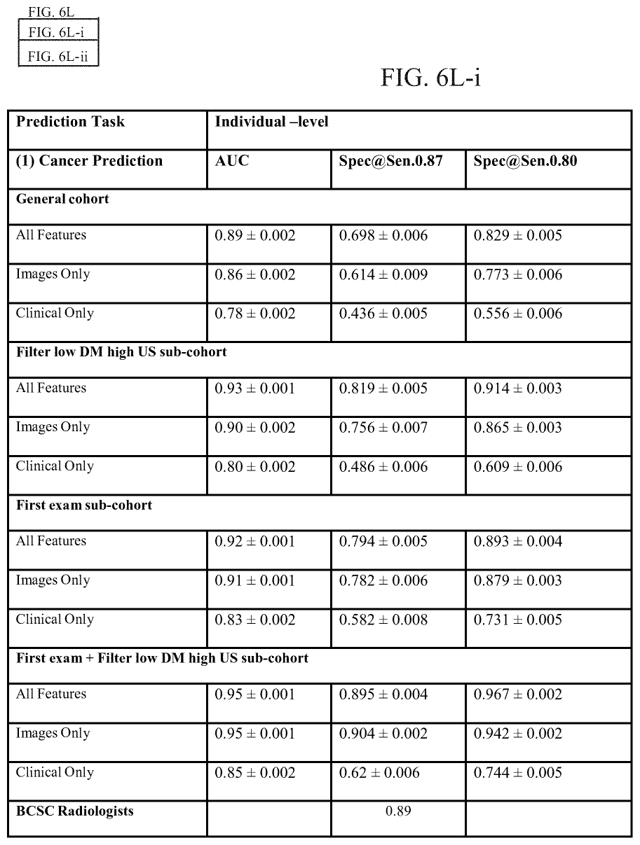

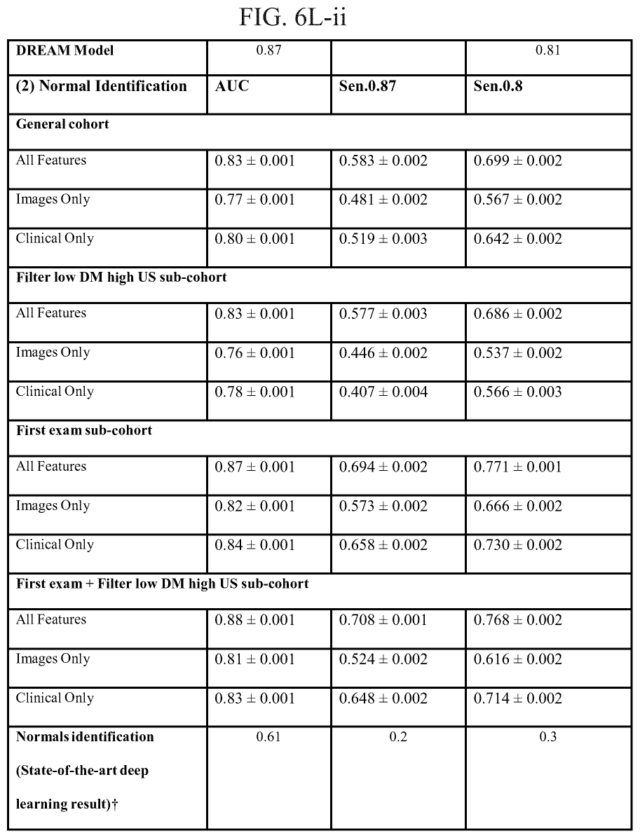

Reference is now made to FIG. 6A, which is a table depicting breakdown to sub-cohorts of the population used in the experimental evaluation, in accordance with some embodiments of the present invention. The table presents train, validation, and test sizes by cohort. The general cohort includes all women that fulfilled the exclusion criteria. ‘Filter low DM high US’ a sub-cohort excluding exams in which DM BI-RADS 1-2 and US BI-RADS≥3. ‘First exam’: a sub-cohort of the general cohort limited to the first DM with and without the US constraint, respectively.

A false negative (FN) was defined as individuals that were assigned final BI-RADS 1-2 but were retrospectively found to have a malignant finding within 12 months from index test.

Outcome Definitions. The study focuses on two clinical tasks for DM screening. First, Prediction of cancer-positive biopsy (referred to herein as Task 1): Following the ACR BI-RADS (e.g., as described with reference to Sickles E A, D'Orsi C J, Bassett L W. American College of Radiology Breast Imaging Reporting and Data System Atlas (ACR BI-RADS Atlas). Reston, Va.: American College of Radiology; 2013 and Lehman C D, Arao R F, Sprague B L, et al. National performance benchmarks for modern screening digital mammography: update from the Breast Cancer Surveillance Consortium. Radiology. 2016; 283 (1):49-58, women were considered to have cancer if the MHS registry/pathology database indicated the diagnosis within 12 months from index test. Positive results included cancer in all stages including ductal carcinoma in-situ (DCIS). All other cases were considered negative. Second, Identifying normal exams (referred to herein as Task 2): Women with BIRADS 1-2 who have had normal follow-up exams for at least two years following index test. Benign and malignant biopsies as well as BI-RADS 3 were not considered normal in this analysis.

Results were reported for both the breast/side-level and the individual level. The first, to better facilitate comparison to other models that evaluate their performance per breast. The latter, to give women a final result in similar fashion to radiologists.

The implementation of the model used in the experimental evaluation is now described. It is noted that the implementation is an example and not necessarily limiting.

The model was designed to output results for the two prediction tasks described above, using as input the DM standard 4-view images and the detailed clinical histories. XGBoost (version 0.81), an open-source Python gradient boosting, as described with reference to Chen T, Guestrin C. XGBoost: A Scalable Tree Boosting System. Proceedings of the 22nd ACM SIGKDD International Conference on Knowledge Discovery and Data Mining—KDD '16. 2016:785-794. doi:10.1145/2939672.2939785 was used as the classifier component of the model to identify a subset of clinical features most contributing to cancer-positive biopsy prediction. The selected sub-set of features were fused into a DNN implementation of the neural network component and trained on each DM image for each of the prediction tasks.

Reference is now made to FIG. 6B, which is a table presenting a description of the IM-DNN neural network component architecture of the model per image, as part of the experimental evaluation, in accordance with some embodiments of the present invention. The table summarizes the details of the neural network component for a single input image with and without clinical information, outputting the dense vectors and softmax value to the next step of the classifier component. †Block 17 or 35, Mixed 5b or 6a—follow the description of InceptionResnetV2. *The sample datum is the input. Otherwise, previous layer is the input.

Reference is now made to FIG. 6C, which is a table presenting clinical features selected by the sub-classifier for input into the neural network component per image, as part of the experimental evaluation, in accordance with some embodiments of the present invention.

For robustness, models were trained on three random 80% partitions of the training set, with and without the subset of clinical features, resulting in six models for each task, which were then used in ensemble average for each task. Following this step, features were extracted from the last fully connected layer, and the estimated probability from the neural network component for each image in each task, and combined with the entire set of clinical features. As such, the probability of a finding at the breast level was acquired from a set comprising the features obtained from both views of the breast (CC and MLO) in both prediction tasks simultaneously, and joined with the entire set of clinical features. The final probability for either cancer-positive biopsy or “normal” identification were estimated using XGBoost implementation of the classifier sub-component. When analyzing at the individual level, the highest probability possible from the two obtained by the model as assigned, as is done in the clinical situation.

Statistical Analysis. Fisher's exact test and t-test were used to estimate the significance of clinical features association with the cancer-positive biopsy outcome. Categorical features were transformed into binary features. Significance of differences between AUCs was estimated using a 95% confidence interval or DeLong test. P-value <0.05 was considered a statistically significant difference.

The percentage of individuals with subsequent biopsy procedures in the cohort was greater than their actual numbers in AMC, as these studies were the first to be transferred from MHS. This did not reflect their distributions in AMC, nor those previously reported in the literature and specifically by the Breast Cancer Surveillance Consortium (BCSC) as described with reference to Lehman C D, Arao R F, Sprague B L, et al. National performance benchmarks for modern screening digital mammography: update from the Breast Cancer Surveillance Consortium. Radiology. 2016; 283 (1):49-58 (0.58% biopsy positive, 2% biopsy negative). To counteract the effect on performance, especially when the benign patients are considered part of the opposite label in both tasks, and to present performance on real-world distributions, a bootstrapping approach was employed. A proportional number of normal, benign, and malignant patients were sampled with replacement according to the proportions of patients in the BCSC, and the corresponding AUC score was calculated. This process was repeated 1,000 times, to obtain mean AUC and a 95% CI, for example, as described with reference to Zadrozny B. Learning and Evaluating Classifiers under Sample Selection Bias. ICML; 2004. The percentages of malignant and benign patients in the literature are reported per population. Inventors estimate that the prevalence of positive and negative biopsies on the breast level is roughly half of the one reported for individuals, for example, as described with reference to Lehman C D, Arao R F, Sprague B L, et al. National performance benchmarks for modern screening digital mammography: update from the Breast Cancer Surveillance Consortium. Radiology. 2016; 283 (1):49-58.

Results—Study Population: The training set included data for 9,611 women, with a mean age of 56±10, body mass index (BMI) 27±5. Of this set, 1,049 women (11%) had a cancer-positive biopsy within 1 year from index test, 1,903 (20%) had a negative biopsy, 247 (2%) were assigned BI-RADS 3 without a subsequent biopsy, and 6,412 (67%) had consistently normal (BI-RADS 1-2) mammograms for at least two years. The held-out test set consisted of 2,548 women, with a mean age of 55±10, BMI 27±5. Of this set, 289 women (11%) had a cancer-positive biopsy, 501 (20%) had a negative biopsy, 70 (3%) were assigned BI-RADS 3, and 1,688 (66%) had normal exams.

Reference is now made to FIG. 6D, which presents patient characteristics in training, validation, and test runs, of the experimental evaluation, in accordance with some embodiments of the present invention. Unless otherwise indicated, data are numbers of patients, and data in parentheses are percentages. As the model made use of the standard 4-view images, the number of images per patients was 4. *Data are mean values and standard deviation in parentheses. ‡ BI-RADS exams without subsequent biopsy procedure within 1 year. †Normal exams are index test exams with final BI-RADS 1-2 with at least two years of normal follow-up.

Reference is now made to FIG. 6E, which is a table presenting statistics by BI-RADS, for the experimental evaluation, in accordance with some embodiments of the present invention. The table presents patient US, biopsy procedure and outcome distribution by mammogram final BI-RADS.

Reference is now made to FIG. 6F, which is another table presenting statistics by BI-RADS, for the experimental evaluation, in accordance with some embodiments of the present invention. The table presents patient biopsy procedure and outcome distribution by final (DM, US) BI-RADS.

A total of 102/13,234 women (<1%) were FN by radiologists. It was preferred to avoid training the model on the data of these patients, so that success in identifying them later could be analyzed. Instead, FN women was inserted in proportional amounts: 31/102 were added to the validation set (to determine the sensitivity operation point threshold) and 71/102 were added to the test set (to reflect their existence in real-world settings). The women's clinical data was integrated with the image information. For each woman in the linked dataset, all available clinical features (total of 1,343) were extracted, including established breast cancer risk factors, for example, as described with reference to Meads C, Ahmed I, Riley R D. A systematic review of breast cancer incidence risk prediction models with meta-analysis of their performance. Breast cancer research and treatment. 2012; 132 (2):365-377. doi:10.1007/s10549-011-1818-2. Women with cancer-positive biopsies tended to be older (p<0.001 Bonferroni adjusted p-value; FIG. 6G) than those without cancer-positive biopsy, they tended to have higher BMI (last measured p<0.003), had more indications of symptoms (lump, nipple retraction or discharge, p<0.004), and had higher incidence of blood tests falling outside the range of expected results (p<0.002). Patients with a cancer-positive biopsy also tended to have a lower incidence of relatives with breast cancer in general (p<0.003) and from first degree in particular (p<0.001).

Reference is now made to FIG. 6G, which is a table presenting association of non-imaging clinical features of interest with cancer-positive biopsy outcomes, for the experimental evaluation, in accordance with some embodiments of the present invention. A complete list of features for the general cohort may be found in FIG. 6H, for first-exam individuals in FIG. 6I. ‘Individuals with cancer-positive biopsy exams’ and ‘Individuals with normal or negative biopsy exams’ show the mean and standard deviation (count and percentage for binary features). p-value is Bonferroni adjusted by the number of features. p-values <0.05 are considered significant.

Reference is now made to FIG. 6H, which is a table presenting a list of non-imaging clinical features association with the cancer-positive biopsy outcome, for the experimental evaluation, in accordance with some embodiments of the present invention. The table presents the complete list of features for the general cohort. ‘Individuals with cancer-positive biopsy exams’ and ‘Individuals with normal or negative biopsy exams’ show the mean and standard deviation (count and percentage for binary features). p-value is prior to Bonferroni adjustment. Rightmost column indicates if significant post adjustment. Features with a signal below 3% were removed from this table. Abbreviations: ind (indicator, refers to binary features), cnt (count, refers to number of occurrences), HRT (hormone replacement therapy), cbc (complete blood count), B (blood), U (urine), TSH (thyroid-stimulating hormone), PT (prothrombin time), HbA1C (Hemoglobin A1C), ALT (alanine transaminase), GPT (glutamic-pyruvic transaminase), AST (aspartate aminotransferase), GOT (glutamic-oxaloacetic transaminase), CRP (C-Reactive Protein), ESR (erythrocyte sedimentation rate), ASCA (Anti-Saccharomyces cerevisiae antibody).

Reference is now made to FIG. 6I, which is a table presenting a list of non-imaging clinical features association with the cancer-positive biopsy outcome in first-exam individual, for the experimental evaluation, in accordance with some embodiments of the present invention. The table presents a complete list of features for the first-exam sub-cohort. ‘Individuals with cancer-positive biopsy exams’ and ‘Individuals with normal or negative biopsy exams’ show the mean and standard deviation (count and percentage for binary features). p-value is prior to Bonferroni adjustment. Rightmost column indicates if significant post adjustment. Features with a signal below 3% were removed from this table. Abbreviations: ind (indicator, refers to binary features), cnt (count, refers to number of occurrences), HRT (hormone replacement therapy), cbc (complete blood count), B (blood), U (urine), TSH (thyroid-stimulating hormone), PT (prothrombin time), HbA1C (Hemoglobin A1C), ALT (alanine transaminase), GPT (glutamic-pyruvic transaminase), AST (aspartate aminotransferase), GOT (glutamic-oxaloacetic transaminase), ESR (erythrocyte sedimentation rate).

Testing of ML-DL Model: All tasks and scenarios were evaluated on several cohorts: (i) General cohort, (ii) ‘Filter low DM and high US’: For some patients, the suspicious finding was only detected in an US exam and not in the mammogram (DM final BI-RADS 1-2, US final BI-RADS≥3). Since some implementations of the model analyze DM images, excluding those led to a sub-cohort that is more applicable to the task at hand. (iii) First-exam only: a sub-cohort limited to the first DM of a woman. (iv) First-exam and filter low DM and high US.

Reference is now made to FIG. 6J, which is a table presenting results on the two tasks on the breast/side-level compared to other deep learning approaches on different subsets of clinical features, without image features, performed as part of the experimental evaluation, in accordance with some embodiments of the present invention. The table presents a sensitivity analyses on subsets of clinical features including: 1) All features except those of symptoms that may lead to a diagnostic exam (lump, nipple discharge and nipple retraction), 2) All features without past density and BI-RADS (from previous radiologist report if exists).

For the table in FIG. 6J, AUC=area under the ROC curve. Each use case displays results for the test set of the general cohort and three sub-cohorts, on a clinical-based analysis. Results reported on the breast/side-level. For each task the results are reported for the test sets of the general cohort and three sub-cohorts. In addition, for each cohort type results are provided for using three subsets of clinical data: 1) all features 2) without features indicating of a symptom 3) without BI-RADS and BD as reported in the most recent radiologist report, if one existed. ‘Filter low DM high US’: a sub-cohort where patients with suspicious finding only seen in an US exam (DM final BI-RADS 1-2, US final BI-RADS≥3) were excluded. † Normals identification state-of-the-art deep learning results were previously obtained by Geras K J, Wolfson S, Shen Y, Kim S, Moy L, Cho K. High-resolution breast cancer screening with multi-view deep convolutional neural networks. arXiv preprint arXiv:170307047.2017.

Reference is now made to FIG. 6K, which is a table presenting results on the two tasks compared to other deep learning models on the breast/side-level, performed as part of the experimental evaluation, in accordance with some embodiments of the present invention.

Reference is now made to FIG. 6L, which is a table presenting results on the two tasks compared on the individual level to results computed by other learning approaches, performed as part of the experimental evaluation, in accordance with some embodiments of the present invention. Results are reported on the individual level. For each task the results are reported for the test sets of the general cohort and three sub-cohorts. In addition, for each cohort type results for using clinical information alone, images alone, and both images and clinical information, are provided. ‘Filter low DM high US’ a sub-cohort excluding exams in which DM BI-RADS 1-2 and US BI-RADS≥3. † Normals identification state-of-the-art deep learning results were previously obtained by Geras K J, Wolfson S, Shen Y, Kim S, Moy L, Cho K. High-resolution breast cancer screening with multi-view deep convolutional neural networks. arXiv preprint arXiv:170307047. 2017.

Results were also compared to existing deep-learning models on the breast level, for example, as described with reference to Katalinic A, Bartel C, Raspe H, Schreer I. Beyond mammography screening: quality assurance in breast cancer diagnosis (The QuaMaDi Project). British journal of cancer. 2007; 96 (1):157 and Howell A, Anderson A S, Clarke R B, et al. Risk determination and prevention of breast cancer. Breast Cancer Research. 2014; 16 (5):446. doi:10.1186/s13058-014-0446-2.

Overall the model described herein that combines images and non-imaging clinical data performed better than other models trained on images alone, or clinical data alone. For the task of malignancy prediction on the general cohort, the model described herein achieved an AUC of 0.91 (95% CI, ±0.002). Other approaches using images alone achieved an AUC of 0.88±0.002, and approaches using clinical data alone achieved an AUC of 0.78±0.003. On the ‘filter low DM and high US’ sub-cohort, the model described herein performed even better (0.94±0.002, 0.91±0.002, 0.81±0.003, respectively). Performance was similar for first-exam individuals (0.94±0.002, 0.93±0.002, 0.85±0.003, respectively), and on a cohort that combined the two constraints—filtering low DM and high US as well as focusing on first-exam individuals (0.96±0.002, 0.95±0.002, 0.86±0.003, respectively).

According to the BCSC (e.g., as described with reference to Lehman C D, Arao R F, Sprague B L, et al. National performance benchmarks for modern screening digital mammography: update from the Breast Cancer Surveillance Consortium. Radiology. 2016; 283M:49-58), the average radiologist performance in screening mammography is 89% specificity at 87% sensitivity. Despite the model's performance being lower than that of an average radiologist (77.3% specificity at 87% sensitivity, 95% CI±0.008), it is noted that for all cohorts the best results are comparable to human radiologists' acceptable range as described in the American national benchmark for screening DM (e.g., as described with reference to Lehman et al.) (75% sensitivity and 88%-95% specificity). Adding clinical features to the analysis improved the AUC by up to 3.17%, and specificity by 6.74% at sensitivity of 87%. As the model output is a probability and not necessarily a final decision, a threshold may be set for a final yes/no decision using the validation set. This was accomplished first, by choosing an operation point of 87% sensitivity (the average radiologist operation point), and second reflecting high specificity (99%). For the average radiologist sensitivity, the model correctly diagnosed 34/71 (48%, 95% CI±0.02) of the FN patients in the test set. Using the second operation point with high specificity, the model obtained 52% sensitivity (95% CI±0.006) overall and correctly diagnosed 11/71 of FN patients in the test set. For the task of “normal” exam differentiation, the model described herein achieved an AUC 0.85±0.001 using both images and clinical information, 0.80±0.001 on images, and 0.80±0.001 on clinical alone. Focusing on first-exam individuals, the results improved (0.88±0.001, 0.84±0.001, 0.85±0.001, respectively). On the ‘filter low DM and high US’ sub-cohort (and the additional one including first-exam individuals), the performance was reduced (0.846±0.001, 0.79±0.001, 0.78±0.001, respectively). Adding clinical features improved the AUC by up to 6.76%, and specificity (at sensitivity 87%) by up to 16.85%. Using an operation point with high sensitivity (99%), the specificity was 22% (95% CI±0.005).

The results for identifying highly probable healthy individuals using only non-imaging clinical data indicate the potential for personalized screening methods, by training models and/or other models on rich existing non-imaging clinical data. The results for both tasks at the extreme operation points, specifically on the false negative test set, exemplify the ability of trained models in guiding treatment of patients, for example, to function as second readers in addition to manual physician analysis.

It is noted that the general cohort, lower incidence of breast cancer family history are reported in women with positive biopsy. This may be explained by the exclusion of past patients with breast cancer from the cohort. This exclusion only affects the number of women with family history that were found to have a cancer-positive biopsy, without affecting the number of women without a cancer-positive biopsy. This counter-intuitive finding is expected to be present in other studies excluding breast cancer survivors. Indeed, Wu et al. as described with reference to Wu Y, Abbey C K, Chen X, et al. Developing a utility decision framework to evaluate predictive models in breast cancer risk estimation. Journal of Medical Imaging. 2015; 2 (4):041005.doi:10.1117/1.JMI.2.4.041005 report similar results. One approach for correcting this selection bias is to limit the cohort to women undergoing their first mammogram, rendering this exclusion criterion moot. Thus, results are reported for first-exam sub-cohorts in addition to the general cohort, where the selection bias was corrected, obtaining improved results (AUC of 0.94±0.002 and 0.96±0.002).

The model was trained on images from Assuta facilities, which use a single mammography vendor (Hologic), and the clinical data originated from Maccabi Health Services (MHS) facilities alone. As such, these results may be validated across different vendors, facilities, and populations around the world. Availability in the clinical data available in different facilities may be expected, however the identification of most contributing features for each prediction task should assist in reproducing these results in other facilities. As such, the accuracy of the model described herein is expected to remain significantly high for other facilities, vendors, and/or populations, and/or for different types of cancer. It is noted that design parameters of the model may be adjusted according to the facility, vendor, population, and/or type of cancer to achieve optimal classification results, for example, variability in the design of the neural network sub-component (e.g., number of layers, design of layers).

Due to the process by which data were transferred from MHS, many patients were excluded on the basis of having a single normal DM without sufficient follow-up to determine that they are indeed normal. On the other hand, many patients with benign findings were introduced into the cohort. This issue is addressed by sampling patients by their real-world distribution. The distinction between screening and diagnostic studies in Assuta is not well-defined; this was addressed by analyzing only the standard views available in screening exams. It is noted that the model described herein may provide a localization of the finding, in addition to a global probability for the entire breast.

Additional details of the described experimental evaluation are now provided.

Methods—Data source: Patients' EHR were extracted from the MHS database. These records include: demographic information, pharmacy records of purchased medications, past diagnoses including previous cancers, treatments and procedures, lab results, biopsy results, and smoking habits. Prior to any examination in an Assuta radiology facility, patients completed a questionnaire (dedicated intake information provided by the patient upon admission to mammography clinic), covering additional data such as: gynecologic history (e.g., age at menarche, number of pregnancies, number of children, menopausal status), personal history of breast cancer, family history of breast and ovarian cancer, self-reported symptoms, and previous procedures. BI-RADS assessments were reported for DM and US separately; past BD and current recommendations were extracted from the radiologist's report. Table S6 lists the set of clinical features used in the experiment. Standard quad-view screening in full-field digital was obtained for each eligible patient. A patient's examination included two standard views of each breast, craniocaudal (CC) and mediolateral oblique (MLO), where all mammograms were acquired using Hologic Dimensions, 1.72 device. A quad-view may be obtained per patient.

Screening policy: As part of AMC's policy, most women (9,799/13,214, 74%) routinely undergo a breast ultrasound (US) exam in addition to DM. Radiologists attempt to complete a BI-RADS 1-5 distinction in a single combined assessment based on both DM and US, if one exists, and BI-RADS 0 is rarely used. Israeli women ages 50-74 are eligible for free breast cancer screening biannually. Performing DM in other scenarios (including early or more frequent screening) requires copay, which is waived in the case of any clinical indication. Such indication appeared for 15.5% (2,048/13,214) of the women. For that reason, using criteria of clinical referral to distinguish true situations of clinical suspicion from screening is irrelevant. Furthermore, only the DM standard 4-view images were analyzed without any additional diagnostic views. Therefore, Inventors concluded that this cohort resembles a screening population much more than a diagnostic one.

The model described herein includes classifier components that for the experimental evaluation were implemented based on the XGBoost implementation of gradient boosting machines (GBM) classifier. First, optionally to identify a small set of clinical features that would be concatenated to the image features as part of the DNN architecture (e.g., 30 features out of the 1,343 available ones). Second, to obtain the final value indicative of likelihood of malignancy, when running the final classification on the two tasks (cancer-positive biopsy prediction, normal identification). At that stage, a single feature vector represents the individual's breast/side-level, as described herein. This feature vector includes the entire set of clinical features, and image features extracted from the neural network component (e.g., DNN) for the two views (CC and MLO), as described herein.

GBM is an ensemble method, including weak learners (shallow decision trees), and is estimated iteratively to result in a stronger learning model. Weak models are added sequentially, as a result of the error of the whole ensemble learnt so far. XGBoost often has an advantage over common classifiers such as logistic regression, as it does not try to fit a linear model, does not require almost any pre-processing of the features prior to training (i.e. standardization, imputation), does not necessarily require prior feature selection to filter out over-correlated features. Simply put, those issues are handled implicitly by the addition of more trees to the sequence.

GBM is known to have two main disadvantages—overfitting and interpretability. Overfitting was avoided herein by tuning the model's hyper-parameters using fivefold cross-validation on the training set. Feature contribution was estimated using the Tree SHAP algorithm, for example, as described with reference to Lundberg, S. M., Erion, G. G., Lee, S. I. Consistent individualized feature attribution for tree ensembles. arXiv:1802.03888. (2018). SHAP estimates feature contributions by calculating the individual impact on the prediction of each feature and for each individual.

Image classification by neural network (NN) component: The neural network component of the classifier was implemented as the DNN described herein. The DNN per image was trained end-to-end. During training the images were cropped to 4096×2048, centered, and resized to 2048×1024. Pixel intensities were normalized to the range [−1.0, 1.0], with no mean centering or any additional standardization. The image was fed into a customized NN based on InceptionResnetV2 feature extractor, with all layers after the “mixed_6a” layer removed, to reduce overfitting and to meet GPU memory budget. After the last convolution, a global max pooling layer was used outputting the feature vector of 1088 elements per sample, as described herein.

The DNN was trained in parallel both with and without non-imaging clinical features. 30 clinical features were selected as input; they were standardized and imputed using a constant negative value (−10). The clinical features were passed through multiple fully connected layers, starting from 30 input features and ending with 256 features. Then, both the 1088 features from the image feature extraction and the 256 features from the clinical data dense layers were concatenated into one feature vector, as described herein. The features were then passed to a fully connected layer of size 256 and followed by a fully connected layer of two classes. At the final layer of the network the activations were mapped to a distribution over the classes (|, θ) by a softmax function. FIG. 6B, presents the details of the architecture.

ReLU was used on all layers except the final classification layer. Batch normalization was used across all layers excluding the final classification layer. The training data was augmented with random affine transformation and color transformation. The weights of the network were initialized using Xavier initialization (e.g., as described with reference to Glorot, X., Bengio, Y. Understanding the difficulty of training deep feedforward neural networks Proceedings of the thirteenth international conference on Artificial Intelligence and Statistics (AISTATS). (2010) and trained end-to-end using Adam (e.g., as described with reference to Kingma, D. P., Ba, J. A. A method for stochastic optimization. International Conference on Learning Representation. arXiv:1412.6980. (2015) with a learning rate of 0.0001. A weight decay L2-norm regularization term of 0.0002 was added. The mini batch size was set to 27 including 3 samples per GPU times 3 GPUs times 3 multi-steps for averaging gradients. Data were sampled by oversampling the smaller classes (benign and malignant biopsies). This resulted in 40 k, 50 k images per epoch, with 13, 3.5, 2 repetitions smaller classes per use case 1, 2, 3 respectively. The entire set of images was shuffled at the beginning of each epoch, and a mini-batch of 27 samples was drawn at each train step. The network was trained up to 40, 10, 6 epochs respectively. The final weights were chosen by monitoring the AUC for a receiver operating characteristic (ROC) plot on the validation set. The total train time was 2 days. Pytorch (0.4, open-source) was used as the deep learning framework. All of the experiments were performed on a computer with a 2 Intel(®) Core(™) i7-5930k 3.50 GHz_8 CPU with 380GB RAM and 8 graphic cards NVIDIA GeForce TITANX 1080Ti.

Results—Integration of clinical features into the DNN: Clinical data were integrated into the images in two ways—by finding a smaller set of clinical features that was concatenated into the imaging features in the final layers of the network, and by wrapping the imaging features extracted from the DNN with clinical images to form a breast/side representation, as described herein. Due to dimensionality issues, not all 1,343 clinical features could be concatenated into the DNN at the first stage. The classifier component, implemented using XGBoost, was used to identify a smaller list of 30 features that contribute to cancer-positive biopsy prediction (AUC of 0.63). Alternatively, Gail et al. score obtained inferior results (AUC 0.54, p<0.001), covering features: age, age at first menstruation, age at first live birth, number of past biopsies, family history of breast cancer. Nonetheless, most Gail et al. features are indeed covered by the features that were identified by XGBoost at this stage, and those that are not, are known to be associated with high risk in other models, for example, as described with reference to Meads C, Ahmed I, Riley R D. A systematic review of breast cancer incidence risk prediction models with meta-analysis of their performance. Breast cancer research and treatment. 132 (2):365-377 (2012) and Wu Y, Abbey C K, Chen X, et al. Developing a utility decision framework to evaluate predictive models in breast cancer risk estimation. Journal of Medical Imaging. 2015; 2 (4):041005. doi:10.1117/1.JMI.2.4.041005. The entire set of clinical features performed better than Gail et al. as well (p<0.004). Adding Gail et al. score as a feature did not significantly contribute to the prediction ability (p>0.05).

Feature contribution analysis: A model was trained without clinical features in the DNN to clearly estimate the features' contribution. The distinction between the full and first-time cohorts is important, as the selection bias in the general cohort may lead to less generalizable prediction (i.e., features such as ‘past_birads_max’ which indicates higher malignancy probability in patients with lower BI-RADS in the past.). In both cohorts, the current indication of a symptom (lump, nipple retraction or discharge) and patient age play an important role. As expected, indications of symptoms have the opposite effect on “normal” differentiation and positive biopsy prediction. The older the patient, the higher the impact on malignancy prediction. If a patient is “too young” it lowers the probability for a normal exam. A high BMI, younger menstruation age, and current use of HRT all contribute positively to malignancy prediction. Breastfeeding, no current use of HRT, and low BMI contribute to “normal” differentiation. A positive indication of family history of breast cancer also seems to contribute to “normal” identification, which was not expected. Possible explanations could be that the population in Israel, and specifically women of Jewish Ashkenazi descent, tend to have a higher incidence of breast cancer in the family in general. Moreover, this feature is self-reported, and therefore could be noisy. Last, it is important to remember that the impact is greatly influenced by the interaction between this feature and all others. Notably, and repeatedly in all the analyses, features representing characteristics of white blood cells (WBC) and the immune system stand out. For example, patients with a positive biopsy prediction tend to have a higher percentage of eosinophils and lower percentage of monocytes; the reverse is true for “normal” differentiation.

Discussion: FIGS. 6E-6F confirm that most mammograms and US exams are indeed normal (BIRADS 1-2). The majority of the population undergo US exams as part of the screening process, even for individuals with mammogram BI-RADS 1-2. Both tables follow the trend that the higher the BI-RADS assessment, the more likely the biopsy results will be malignant. Not surprisingly, many of the clinical features that the model identified as most contributing to the prediction are known risk factors for breast cancer. Older age, family history of breast cancer and higher BMI were reported in the past to increase breast cancer risk, for example, as described with reference to Meads C, Ahmed I, Riley R D. A systematic review of breast cancer incidence risk prediction models with meta-analysis of their performance. Breast cancer research and treatment. 132 (2):365-377 (2012). Breastfeeding (currently, and the number of children breastfed in total) is known to reduce risk, for example, as described with reference to Breast cancer and breastfeeding: collaborative reanalysis of individual data from 47 epidemiological studies in 30 countries, including 50 302 women with breast cancer and 96 973 women without the disease. The Lancet (2002). Hypothyroidism was also reported to be related to breast cancer, for example, as described with reference to Hercbergs A, Mousa S A, Leinung M, Lin H-Y, Davis P J. Thyroid Hormone in the Clinic and Breast Cancer. Hormones and Cancer. 9 (3):139-143 (2018).

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant anatomical images, non-imaging clinical parameters, classifiers, neural networks, and models will be developed and the scope of the terms anatomical images, non-imaging clinical parameters, classifiers, neural networks, and models are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of selecting patients for treatment, comprising:
feeding at least one anatomical image of a patient depicting a target tissue, and a plurality of non-imaging clinical parameters of the patient into at least one neural network component of a model;
outputting by the at least one neural network component, an intermediate vector storing a plurality of embedding values computed for the at least one anatomical image, a plurality of values outputted by a dense layer of the at least one neural network component in response to an input of at least some of the non-imaging clinical parameters, and an intermediate value indicative of likelihood of malignancy for the target tissue;
feeding into a classifier component of the model, a feature vector created from the intermediate vector and the plurality of non-imaging clinical parameters; and
selecting patients for treatment according to an indication of likelihood of malignancy in the target tissue outputted by the model.

2. The method of claim 1, wherein treatment comprises a biopsy of the target tissue.

3. The method of claim 1, wherein selecting comprises tagging a record of the patient with an indication of recommendation for treatment, for patients having a value indicative of likelihood of malignancy above a threshold.

4. The method of claim 1, wherein the at least one neural network comprises a first and second subset, wherein the at least one anatomical image and the plurality of non-imaging clinical parameters are fed into the first subset and the at least one anatomical image is fed into the second subset,
wherein the intermediate vector stores outputs of the first and second subsets, wherein the first subset outputs the plurality of embedding values, the plurality of values outputted by the dense layer, and the intermediate value, and the second subset outputs a second plurality of embedding values computed for the at least one anatomical image and a second intermediate value indicative of likelihood of malignancy for the target tissue.

5. The method of claim 1, wherein the at least one anatomical image comprises a plurality of anatomical images, wherein each one of the plurality of anatomical images is fed into the at least one neural network component, wherein outputs of the at least one neural network for the plurality of anatomical images are aggregated to compute the intermediate vector.

6. The method of claim 1, wherein each of the plurality of anatomical images depicts a respective unique image planes of the target tissue.

7. The method of claim 1, wherein the at least one anatomical image comprises a plurality of anatomical images each fed into the at least one neural network component, the feature vector further includes a plurality of metadata values computed for pairs of the plurality of anatomical images according to relationships between respective likelihood of malignancy computed by the at least one neural network component.

8. The method of claim 7, wherein the relationships are computed for a first and second image of each pair of the plurality of anatomical images, selected from the group consisting of: likelihood of malignancy for the first image divided by a sum of likelihood of malignancy for the first image and likelihood of malignancy for the second image, absolute value of a difference between likelihood of malignancy for the first image and likelihood of malignancy for the second image, and maximum of the likelihood of malignancy for the first image and likelihood of malignancy for the second image.

9. The method of claim 1, wherein the intermediate vector is computed as an output of a last fully connected layer that receives a concatenation of an output of a sub-component of the at least one neural network that is fed the at least one anatomical image, and an output of the dense layer of the at least one neural network that is fed at least some of the non-imaging clinical parameters.

10. The method of claim 1, wherein the intermediate value is outputted by an output layer of the at least one neural network that is fed the at least one anatomical image and the plurality of non-imaging clinical parameters.

11. The method of claim 1, wherein the at least some of the non-imaging clinical parameters are selected according to a training dataset of a plurality of sample patients, according to a statistical correlation between each non-imaging clinical parameter and a ground truth indicative of a diagnosis of malignancy.

12. The method of claim 1, wherein the at least one neural network comprises an ensemble of a plurality of neural networks each differing by at least one neural network parameter, wherein the at least one anatomical image comprises a plurality of anatomical images each fed into each neural network of the ensemble, wherein the intermediate vector is computed as an aggregation of the plurality of embedding values computed by the ensemble and an aggregation of a plurality of values outputted by each respective dense layers of the ensemble.

13. The method of claim 1, wherein the non-imaging clinical parameters exclude an external manual and/or automatic analysis of the at least one anatomical image.

14. The method of claim 1, wherein the indication of likelihood of malignancy in the target tissue comprises indication of likelihood of malignancy in breast tissue.

15. The method of claim 14, wherein the non-imaging clinical parameters are selected from the group consisting of: demographics, age, last body mass index, maximum body mass index, last body mass index class, maximum body mass index class, gynecological history, age at first menstruation, age at last menstruation, indication of postmenopausal, number of menstruation years, pregnancies count, past pregnancies, indication of is breastfeeding, number of children breastfed, indication of current use of hormone replacement therapy, cancer history, family breast cancer first degree, family breast or ovarian cancer, number of relatives with breast or ovarian cancer, minimum age in family for cancer, any personal cancer history, symptoms, lump complaint by woman, bilateral lump complaint by woman, lump complaint by woman in the past, pain complaint by woman, bilateral pain complaint by woman, breast radiology history, past number of breast imaging encounters, past breast density, past final BIRADS assessment DM left, past final BIRADS assessment DM right, past final BIRADS assessment US left, past final BIRADS assessment US right.

16. A method of training a model used for selecting patients for treatment, comprising:
training at least one neural network component of the model for outputting an intermediate value indicative of likelihood of malignancy for a target tissue of a target patient in response to an input of at least one anatomical image depicting a target tissue of the target patient, and at least some non-imaging clinical parameters of the target patient, according to a training dataset storing, for each of a plurality of sample patients, a ground truth indication of malignancy, at least one anatomical image depicting the target tissue, and value for the plurality of non-imaging clinical parameters;
creating an intermediate training dataset storing a respective feature vector for each of the plurality of sample patients, wherein each feature vector is created from a respective intermediate vector and the plurality of non-imaging clinical parameters for the respective sample individual, wherein each respective intermediate vector stores a plurality of embedding values computed for the at least one anatomical image of the respective sample individual and a plurality of values, outputted by a dense layer of the trained at least one neural network component in response to an input of at least some of the non-imaging clinical parameters of the respective sample individual, and an intermediate value indicative of likelihood of malignancy for the target tissue of the sample individual;
training a classifier component of the model according to feature vectors stored in the intermediate training dataset and corresponding ground truth indications of malignancy; and
providing the model including the trained at least one neural network component and the trained classifier component for selecting patients for treatment according to a computed indication of likelihood of malignancy in a target tissue of a target patient outputted by the model.

17. The method of claim 16, further comprising:
defining the at least some of the non-imaging clinical parameters based on the training dataset by computing a statistical correlation between each non-imaging clinical parameter and ground truth indication of malignancy, and selecting the at least some of the non-imaging clinical parameters according to a requirement of the statistical correlation.

18. A system for selecting patients for treatment, comprising:
at least one hardware processor executing a code for:
feeding at least one anatomical image of a patient depicting a target tissue, and a plurality of non-imaging clinical parameters of the patient into at least one neural network component of a model;
outputting by the at least one neural network component, an intermediate vector storing a plurality of embedding values computed for the at least one anatomical image, a plurality of values outputted by a dense layer of the at least one neural network component in response to an input of at least some of the non-imaging clinical parameters, and an intermediate value indicative of likelihood of malignancy for the target tissue;
feeding into a classifier component of the model, a feature vector created from the intermediate vector and the plurality of non-imaging clinical parameters; and
selecting patients for treatment according to an indication of likelihood of malignancy in the target tissue outputted by the model.

19. The system of claim 18, further comprising a code for:
training the at least one neural network component of the model for outputting the intermediate value indicative of likelihood of malignancy for the target tissue of the target patient, according to a training dataset storing, for each of a plurality of sample patients, a ground truth indication of malignancy, at least one anatomical image depicting the target tissue, and value for the plurality of non-imaging clinical parameters;
creating an intermediate training dataset storing a respective feature vector for each of the plurality of sample patients, wherein each feature vector is created from a respective intermediate vector and the plurality of non-imaging clinical parameters for the respective sample individual, wherein each respective intermediate vector stores a plurality of embedding values computed for the at least one anatomical image of the respective sample individual and a plurality of values, outputted by a dense layer of the trained at least one neural network component in response to an input of at least some of the non-imaging clinical parameters of the respective sample individual, and an intermediate value indicative of likelihood of malignancy for the target tissue of the sample individual; and
training the classifier component of the model according to feature vectors stored in the intermediate training dataset and corresponding ground truth indications of malignancy, wherein the model includes the trained at least one neural network component and the trained classifier component.
